(19) United States
(12) Patent Application Publication
(10) Pub. No.: US 2026/0258342 A1
(43) Pub. Date: Sep. 3, 2026

(54) METHODS AND ARRANGMENTS FOR PROCESS ANALYTICAL TECHNOLOGY MICROBIAL TESTING SKID

(71) Applicant: Arranta Bio Holdings, LLC, Watertown, MA (US)

(72) Inventors: Edita Botonjic-Sehic, Watertown, MA (US); Syed Kaschif Ahmed, Watertown, MA (US); Mark Jandreski, Watertown, MA (US); Aaron Cowley, Watertown, MA (US)

(21) Appl. No.: 19/552,738

(22) Filed: Feb. 27, 2026

Related U.S. Application Data
(60) Provisional application No. 63/764,798, filed on Feb. 28, 2025.

(51) Int. Cl. C12M 1/36; C12M 1/06; C12M 1/34; G05B 13/04

(52) U.S. Cl. CPC C12M 41/48; C12M 27/02; C12M 41/36; G05B 13/042

(57) ABSTRACT

Embodiments provide a process analytical technology microbial testing (pat box) skid (mPBS) as a modular apparatus comprising at least one process analytical instrument and a controller adapted for fluid communication with a operation of a purified water, pharmaceutical or biopharmaceutical manufacturing system used for measuring CQAs of a biomanufacturing or purified water process, systems comprising the modular apparatus, and related compositions and methods. Logic may establish monitoring for a Critical Quality Attribute (CQA) in real-time and one or more parameters for collecting measurement data from an in-line instrument, wherein the measurement data is analyzed to determine CQAs with one or more models. The logic may collect, based on the one or more parameters, the measurement data in real-time from the in-line instrument. The logic may process the measurement data from the in-line to generate real-time transformed data for the CQA and output trending data for the CQA.

### BACKGROUND

In pharmaceutical manufacturing, water is widely used either as a raw material, as an ingredient, or as a final product. Water is also used for washing and rinsing equipment or for preparing disinfectants and detergents. These applications require pharmaceutical-grade water, which has been through a chemical purification step. Purification is undertaken so that the water is free of substances that might cause interaction with drug substances, as well as to obtain water of an appropriate microbiological standard.

In water systems microbiological risks are present and risks can arise through poorly maintained water generation systems, through poorly designed distribution networks where ineffective and non-sterile tubing management can lead to contamination. Problems in water systems are caused by presence of microorganisms and varied in their ability to survive and grow under different conditions. Therefore, monitoring pharmaceutical-grade water systems for bioburden is important.

Concerns are two primary types of water: purified water and water for injections (WFI). Both types should be assessed for bioburden, while WFI also requires bacterial endotoxin testing. Additional concerns also include conductivity of water, pH, total organic carbon, and additional chemical tests such as nitrate in some instances specific for highly purified water. For context, there are several different categories of purified water for pharmaceutical purposes. There are also various testing requirements depending on the regulating body. For example, FIG. 3 outlines the purified water and related sub-type quality requirements according to the United States (US) Pharmacopeia (USP). For refence and context, FIG. 3 also overlays the required testing for the quality criteria on top of tests required for drinking water according to the US Environmental Protection Agency (EPA) to show common tests between USP grade purified water and drinking water. For additional context, the European Medicines Agency (EMA) sets quality guidelines for pharmaceutical water, and references multiple types of water including WFI, Purified Water, Water for preparation of extracts, and potable water, see FIG. 5. Alongside the EMA water guidelines, the European Pharmacopoeia (Eu. Ph.) sets quality standards for WFI, Sterilized WFI, Purified Water, and Water for preparations of extracts, see FIG. 4. Similar FIG. 3, FIG. 4 also shows the Potable Water standards set by the European Council Directive that overlap with the tests required by the Eu. Ph.

In addition to testing water, buffer solutions and drug substance and drug product materials also require microbial and endotoxin testing prior to release approval. Drug and buffer manufacturing require several steps and/or unit operations where open processing might be performed. While this open processing might be conducted in a classified clean space, there is still a risk that bioburden or microbial contamination can occur. Whether the bioburden source is from raw materials, operator handling, or environmental factors, there are several possible entry points. It is essential in many circumstances to control bioburden to minimal level and in some circumstances ensure it is removed all together where the material is then tested for sterility (no growth in the conventional plate-based method). Microbial enumeration or bioburden testing is essential in the buffer and drug manufacturing process to ensure microbial contamination is controlled.

During the manufacture of biopharmaceutical raw materials such as reagents or buffers as well as drug material, unit operation systems are used that need to clean the product contact surfaces prior to running the system again before another batch of material is processed through the system. At a high level, this cleaning process usually entails pumping or soaking the product contact surfaces with cleaning solution such as high pH caustic buffer, detergent, and/or surfactants. Equipment that does not need to be moved or uses its own pumps and piping to clean itself can be cleaned in place (CIP). Equipment that needs to either removed from larger equipment or moved to another area to be cleaned is called cleaning out of place (COP). After a certain contact time, the cleaning solution is rinsed off the product contact surface, typically with purified water. Before or during cleaning validation activities, the water used to rinse the cleaned surfaces (otherwise known as rinsate) is tested for various quality attributes including those like water for injection such as conductivity, pH, TOC, bioburden, and endotoxin, to ensure the product contact surface was adequately cleaned as well as ensure the cleaning buffer solution was thoroughly and adequately rinsed away to a certain degree. While pH and conductivity may be routinely tested in-line, TOC, bioburden, and endotoxin are typically tested off-line in a QC lab.

Microbiological testing using conventional culture-based methods is less accurate and the results are obtained slowly, taking several days to complete the tests and results. While rapid microbiological methods are gradually being implemented (such as ATP bioluminescence techniques, or the use of fluorescent DNA-specific dyes) most microbiological assessments of pharmaceutical grade water are reliant upon cultural methods. In turn, bioburden results are not available until several days have elapsed. This can ultimately cause failures in the process and out of specification situations. Rapid measurements of bioburden on the scale of minutes or a few hours has the potential to make a significant shift in how purified water systems are monitored as well as how biological product production systems characterize bioburden containment and clearance. RMM has the potential to save the pharmaceutical industry significant costs in down time by optimizing sanitization cycles, labor costs, and material costs. Also of significance is the ability to move from one unit operation to the next or to release from one batch to the next in a matter of hours as opposed to days or weeks. For context the USP <61> microbial enumeration test typically takes at least 5 days to incubate the test media and report results. The USP <71> sterility test method typically takes at least 14 days to incubate and report results.

Another common test among monographed purified waters in Bacterial Endotoxin testing. Bacterial endotoxins are lipid polysaccharides (LPS) found in the outer cell wall of gram-negative bacteria. LPS is released when these gram-negative bacteria are lysed. Endotoxin is also a pyrogen, a fever causing agent that poses a safety risk to humans, both in water sources and drug products. Beyond fever, endotoxin can cause a range of adverse reactions in humans such as organ failure, septic shock, and potentially death. Therefore, it is imperative and required by the USP and Eu. Ph. For monographed waters to test for endotoxin routinely in packaged purified water and thus manufacturers will monitor endotoxin levels in their purified water generation systems.

The most commonly accepted endotoxin test by the United State Food and Drug Administration (FDA) is the Limulus Amoebocyte Lysate (LAL) test using a gel-clot method to examine the presence of endotoxin. Multiple variants of the LAL test technique include turbidimetric and chromogenic methods. The LAL test is most commonly an off-line test performed in an analytical lab. Moving towards an at-line or even on-line endotoxin test method would save time and cost in reducing labor costs, material costs, and equipment down time. At-line methods may involve some human intervention for monitoring without feedback control for optimization.

Biomanufacturing relies on analytical testing of critical quality attributes (CQAs) between unit operation to ensure drug substance (DS) and drug product (DP) meet industry and regulatory standards. Analytical testing can be performed using off-line, at-line, on-line, and/or in-line methods. As process analytical technologies advance, more instruments are developed that can measure CQAs using in-line methods. The benefit of in-line methods is that it can measure CQAs in real-time. Real-time measurements do not require human intervention and can be automated to measure according to process schedule. On the other hand, some real-time measurements require post-processing of data and modelling to obtain CQAs. An example of these techniques is spectroscopic instruments. Spectroscopic instruments produce an array of data per measurement that is called spectra. To convert spectra to CQAs, signal processing and multivariate modelling techniques are employed. These data analysis techniques are often called chemometrics. Chemometrics requires special commercial software or programming languages to be implemented on real-time data generated by process analytical technologies. Therefore, to measure CQAs in real-time, beside appropriate in-line instruments, data analysis platforms are required to process data.

Biomanufacturing industry has come up with solutions to manage in-line process analytical technology (PAT) and chemometrics software – PAT management software. These solutions are provided by the private sector and often are inflexible toward biopharmaceutical companies process requirements as these solutions are designed to be a general purpose for all the biopharmaceutical industry.

Commercial PAT management software requires adaptation of new instruments and technologies that can be time-consuming and costly. Besides, they are designed to execute chemometrics models using commercial software which require additional cost and maintenance. Commercial software excels at one type of model and is limited to other types of models. With the recent advances in machine learning models and artificial intelligence, open-source programming languages such as Python are becoming more attractive in the biopharmaceutical industry. Although some commercial PAT management software added Python capability, when it comes to practice, incorporating Python models is cumbersome.

Commercial PAT management software has three main functions. The first main function is the chemometrics, which is basically a toolbox of algorithms that can be used to develop models for instruments. The second main function is adaptation, which includes some adapters for instruments. The third main function is data handling.

Data handling is a central database of in-line PAT measurements. Commercial software provides this feature, however, accessing data is limited to one batch at a time and biopharmaceutical companies will need to develop additional data solutions should they desire to perform additional data analysis. For example, to perform a batch-to-batch comparison, data export and staging from commercial software is a cumbersome and manual task.

### BRIEF SUMMARY

An embodiment of a method, apparatus, system, and computer-readable medium may be described herein. In one aspect, an apparatus of a process analytical technology (PAT) microbial testing skid (mPBS) to capture measurement data for a Critical Quality Attribute (CQA) in real-time includes a vessel for holding a volume of a fluid of a process, one or more flow cells or immersible probes coupled with one or more instruments to generate the measurement data related to the CQA from the fluid of the process, and a PAT box cabinet. The PAT box cabinet may include: the one or more instruments, an interface for receiving the measurement data, a PAT management stack comprising layers of software to manage measurements by the one or more instruments, and a first controller to execute the layers of software of the PAT management stack and to send the measurement data in a raw or processed form to a second controller to determine the CQA.

The apparatus may further include the second controller, where the second controller includes processing circuitry to execute a model to transform the measurement data in real-time and to output transformed data and trending data related to the CQA. The one or more instruments may include up to 10 instruments or more.

In some embodiments, the apparatus may also include the vessel or tank with a volume between 100 milliliters and 100,000 liters. The CQA may include a volume of a substance, a concentration of the substance, or a mass of the substance in a fluid or material of the process. In some embodiments, the PAT box cabinet or the vessel may cause the fluid to recirculate through the one or more flow cells and inline probes of the immersible probes to monitor the process and provide real time analytics. In some embodiments, the PAT box cabinet or the vessel may cause the fluid to recirculate through on-line flow cells of the one or more flow cells and sample small aliquots for at-line testing.

In some embodiments, the apparatus may also include an automated sampling system to collect samples of the fluid for at-line instruments or off-line instruments such as a Modular Automated Sampling Technology (MAST). In some embodiments, the PAT management stack includes one or more modules, where one of the one or modules includes a calibration and signal processing module to calibrate at least one of the one or more instruments. Other technical features may be readily apparent to one skilled in the art from the following figures, descriptions, and claims.

In one aspect, the second controller comprise an advanced processing controller (APC) that includes PAT software to enable concurrent operation of multiple instruments and models. In one aspect, the apparatus may also include a human-machine interface (HMI) to present, in real-time, the transformed data and the trending data related to the CQA. In one aspect, the apparatus may also include one or more of, or a combination of instruments includes a rapid microbial method (RMM) analyzer, endotoxin detector, a Total Organic Carbon (TOC) analyzer, a pH meter, conductivity sensor, temperature sensor, and an index of refraction (IoR) system.

In some embodiments, the apparatus may also include a vessel that is a stirred tank reactor. The apparatus may also include at-line instruments or off-line instruments such as an endotoxin detector. In some embodiments, the calibration and signal processing module is pre-calibrated or automatically calibrated to perform data processing to digitally process the measurement data. In some embodiments, the calibration and signal processing module includes hardware including a flow kit to calibrate or recalibrate at least one of the one or more instruments. In some embodiments, the second controller includes an advanced processing controller (APC) to access a library of models to identify and execute one or more models for at least one of the one or more instruments.

In some embodiments, the APC includes a chemometrics model and processing circuitry to perform chemometrics to process the measurement data for the at least one of the one or more instruments to transform the measurement data to determine the CQA and trending data related to the at least one of the one or more instruments. In some embodiments, the APC includes or has access to a data storage device for storing the measurement data, the transformed data, and the trending data in a centralized database of PAT measurements and model outputs. In some embodiments, the APC includes PAT software to establish one or more parameters for at least one of the one or more instruments, where the one or more parameters comprise commanding acquisition parameters or measurement configurations for an in-line instrument.

In some embodiments, the PAT software includes or has access to a simulator model to execute in parallel with the process to simulate the process. In some embodiments, the simulator model simulates the measurements by the one or more instruments. In some embodiments, the APC includes PAT software to perform measurement sequencing, the measurement sequencing including acquiring measurement data, monitoring measurement status, raising errors if measurements fail, storing raw measurement data, transferring raw measurement data to a model execution web app, receiving transformed data and model outputs from the model execution web app, and storing transformed data and model outputs.

In some embodiments, the APC includes PAT software to enable execution of Python models in a model execution web app. In some embodiments, the model execution web app receives the data from the measurement sequencing and runs Python codes to process measurement data. In some embodiments, the model execution web app executes any Python models that can handle real-time data. In some embodiments, one or more of the Python models comprise pre-treatment methods including interpolating functions, derivation, and normalization; multivariate models including principal component analysis (PCA) and partial least square (PLS) regression; and hybrid models by combining mechanistic equations and the multivariate models. Other technical features may be readily apparent to one skilled in the art from the following figures, descriptions, and claims.

Real-time monitoring is provided to reduce risk in pharmaceutical water generation systems and biopharmaceutical manufacturing systems. Emphasis is also placed on system design and control. Modeling and Advanced Monitoring and Control apparatus connects with each PAT sensor. The Digital Architecture will be able to do predictive maintenance, alarming, and continuous monitoring via the HMI. Predictive maintenance modeling for these systems in biopharmaceuticals typically includes modeling and monitoring equipment such as pumps, heat exchangers, filters, and storage tanks. Critical components like UV disinfection units, reverse osmosis membranes, and distillation units can also be modeled and tracked for signs of wear, fouling, or performance degradation. This holistic modeling and monitoring ensure all elements of the system operate efficiently, safeguarding the water purity and preventing costly failures.

### BRIEF DESCRIPTION OF THE SEVERAL VIEWS OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. In the figures, identical or nearly identical or equivalent elements are typically represented by the same reference characters, and similar elements are typically designated with similar reference numbers, with redundant description omitted. For purposes of clarity and simplicity, not every element is labeled in every figure, nor is every element of each embodiment shown where illustration is not necessary to allow those of ordinary skill in the art to understand the disclosure.

To easily identify the discussion of any particular element or act, the most significant digit or digits in a reference number refer to the figure number in which that element is first introduced.

FIG. 1 illustrates an example of batch processing.

FIG. 2 illustrates an example of continuous processing in accordance with one embodiment.

FIG. 3 illustrates an example schematic of a microbial pat box skid (mPBS) with a process stirred tank reactor in accordance with one embodiment.

FIG. 4 illustrates an example of a mPBS in accordance with one embodiment.

FIG. 5 illustrates an example of a system for a mPBS in accordance with one embodiment.

FIG. 6 illustrates another example of a system for a mPBS in accordance with one embodiment.

FIG. 7 illustrates another example of a mPBS in accordance with one embodiment.

FIG. 8 illustrates a testing matrix for monographs per the Environmental Protection Agency.

FIG. 9 illustrates a testing matrix for monographs for the European Pharmaceutical monographs.

FIG. 10 illustrates a tabulated summary of the EMA Guidelines on the Quality of Water for Pharmaceutical Use.

FIG. 11 illustrates a process in accordance with one embodiment.

FIG. 12 illustrates another example of a mPBS in accordance with one embodiment.

FIG. 13 illustrates an mPBS situated downstream of a water for injection (WFI) generation skid and/or WFI distribution drop in accordance with one embodiment.

FIG. 14 illustrates an example of a process flow diagram (PFD) of a mPBS in accordance with one embodiment.

FIG. 15 illustrates an example of a schematic showing the mPBS connected to the outlet manifold of a buffer dilution skid in accordance with one embodiment.

FIG. 16 illustrates another example of a process flow diagram (PFD) of a mPBS in accordance with one embodiment.

FIG. 17 illustrates an example of results of several samples of water from a mPBS ranging from ultra purified to water that is likely contaminated with microbial material in accordance with one embodiment.

FIG. 18 illustrates an example of a PFD of an aspect of the mPBS ecosystem in accordance with one embodiment.

FIG. 19 illustrates another example of a PFD of an mPBS in accordance with one embodiment.

FIG. 20 illustrates another example of a PFD of an mPBS in accordance with one embodiment.

FIG. 21 illustrates an example of results of RNA drug substance in-process material in accordance with one embodiment.

FIG. 22 illustrates another example of a PFD of an mPBS in accordance with one embodiment.

FIG. 23 illustrates another example of a PFD of an mPBS in accordance with one embodiment in accordance with one embodiment.

FIG. 24 illustrates another example of a PFD of an aspect of the mPBS ecosystem in accordance with one embodiment.

FIG. 25 illustrates an example of a computer system in accordance with one embodiment.

FIG. 26 illustrates an example of a storage medium in accordance with one embodiment.

FIG. 27 illustrates an example of a computing platform in accordance with one embodiment.

### DETAILED DESCRIPTION

The disclosure provides a modular and compact apparatus for deploying process analytical technology (PAT) in a water generation, buffer manufacturing, or drug manufacturing process for measuring quality attributes specific to purified, formulated buffers, and in-process bulk drug material referred to herein as a Microbial PAT Box Skid (mPBS). The mPBS may be a single engineered skid with a PAT Box that houses one or more PAT instruments. and one or more hold tanks or vessels. The mPBS may come with a hold tank or vessel, for recirculation/release purposes, and an adequate number of input-output (IO) ports so that data and information can readily flow back in forth to multiple control panels and/or software. In some embodiments, the hold tank or vessel may be as small as 100 milliliters (ml) and as large as 100,000 liters. In some embodiments, the hold tank or vessel may be between 1 liter and 10,000 liters. In further embodiments, the hold tank or vessel may be between 10 liters and 1,000 liters.

The product may recirculate between the hold vessel and the PAT Box that has a number of PAT tools and/or instruments, from as few as 1 to 10 or more, to take measurements via flow cells and/or via immersible probes. The product may alternately be passed directly from one unit operation to the next via the PAT Box or be sampled at-line for the PAT Box. Data from the at-line instruments may be analyzed by a “smart controller” or computer such as an advanced processing controller (APC) to determine if the batch passes or fails via Real Time Release Testing (RTRT).

The mPBS systems may specifically house PAT equipment that either measures microbial growth, and/or monitors quality attributes that are related to microbial growth, and/or measure quality attributes related to drug product safety such as microbial enumeration counts, endotoxin detection, total organic carbon (TOC), pH, and/or conductivity.

The mPBS systems can be installed between each unit operation, whether that be between a bioreactor and a centrifuge, a chromatography skid and TFF system or any two pieces of bioprocessing equipment that are known by those skilled in the art. The mPBS systems may also monitor purified water generation and/or distribution systems, buffer production systems, and/or Clean-in-Place rinsate. In addition, the mPBS systems may be mobile units that can be plugged in and played along a manufacturing process, depending upon the number and type of PAT instruments that are installed into a specific mPBS system.

Embodiments may generally provide methods, apparatuses, systems, and articles of manufacture for mPBS that brings PAT analytics to operations in a purified water generation system, buffer formulation process, and/or drug manufacturing process or system enabling measurement of water, buffer, and drug substance or product critical quality attributes (CQAs) to be monitored, controlled and released or rejected. The mPBS may provide real-time analysis and trending for real-time monitoring of CQAs for in-line instruments and at-line instruments. Embodiments may configure in-line instruments, provide machine learning models such as multivariate modelling, and provide visualization and analytics for outputs of machine learning models. Some embodiments may provide machine learning models to specifically target CQAs. For instance, some embodiments may include models to target specific molecules to provide concentrations or other CQAs and trends in real-time.

The term CQA is used in its customary sense to refer to a physical, chemical, or biological property or characteristic that should remain within a predetermined limit, range, or distribution to ensure the desired quality. Biomanufacturing runs are expensive due to the high cost of raw materials. Incorporating a mPBS into a continuous water monitoring process, buffer manufacturing, and/or drug manufacturing process provides a significant reduction in time and cost. The reduction of cost comes from reducing the labor and consumables and materials needed for off-line testing. The reduction of time comes from reducing the turnaround time for testing results and reducing time of the purified water system. Having real-time results of CQAs for a water system can better characterize the efficacy of cleaning cycles and reduce down-time by creating more efficient process and cleaning procedures. Regarding buffer and drug manufacturing, reduction of time comes from less time spent waiting for off-line results which can range from 5-14 days where in-process bulk material may need to be stored either on the floor or in cold storage where it takes up valuable space. Turning around microbial results in a matter of minutes or hours as opposed to days frees up manufacturing spaces to continue production on other products thereby making the facility more productive.

In many embodiments, the mPBS systems may, advantageously, reduce the cost of real-time monitoring compared to current commercial solutions as they often require the purchase of multiple licenses. Besides, the mPBS systems may be supported immediately instead of communicating to multiple vendors in case of commercial solutions. This advantageously reduces the time of real-time monitoring implementation and support.

In one aspect, provided are methods for designing and/or optimizing a purified water generation process, buffer manufacturing process, and/or drug manufacturing process utilizing one or more mPBS systems as described herein to identify critical control points (CCPs), i.e., the critical unit operations at which product quality attributes should be monitored for real-time testing and release.

The mPBS system described herein may be used in various biopharmaceutical manufacturing processes including but not limited to buffer solutions, raw material reagents, monoclonal antibodies, antibody drug conjugates, vaccines, including RNA vaccines, RNAi, enzymes, peptides, cell therapies, viral vectors including adeno-associated virus (AAV) vectors and lentivirus vectors, and other gene therapy modalities.

The mPBS system may comprise an appropriate model based on underlying data and assumptions for a given attribute, or CQA. An attribute may include but is not limited to, for example, conductivity, bioburden, endotoxin, TOC, conductivity, temperature, and pH. The model may be a mechanistic model or a data-driven model, or a hybrid model that combines aspects of both mechanistic and data-driven models. A mechanistic model refers to a model based on energy and mass balances and relationships that have been mathematically elucidated based on a full mathematical understanding of the process and may also be referred to as “white‐box models”. Data‐driven models are mathematical models based solely on the statistical relationships between data, primarily data obtained or derived from online sensors and offline analysis. Data‐driven models are not based on biophysical relationships and may also be referred to as “black‐box models.” Hybrid models combine mechanistic and data‐driven models.

In some embodiments, the mPBS system may comprise a large model to handle multiple attributes or all attributes associated with a one or more unit operation (Unit OP). In other embodiments, the mPBS system may comprise multiple models, each model for a single attribute or process or two or more attributes.

The controller may also include algorithms to measure the relationship between variables using correlation analysis which relies on establishing correlations between sensor signals, process parameters, and quantity and quality parameters which may be measured offline. For example, the extent of the linear relationship is determined using a Pearson's correlation. Other methods are available to measure nonlinear relationships, for example, Spearman's rank correlation, which is a nonparametric measure of rank correlation reporting the statistical relationship between the rankings of two variables.

In some embodiments, the controller may include algorithms for carrying out one or more statistical methods selected from multiple linear regression (MLR), partial least squares regression (PLS), structured additive regression (STAR), random forest (RF), support vector machines regression (SVM), neural networks (NNs), deep learning (DL), and Gaussian process regression (GPR).

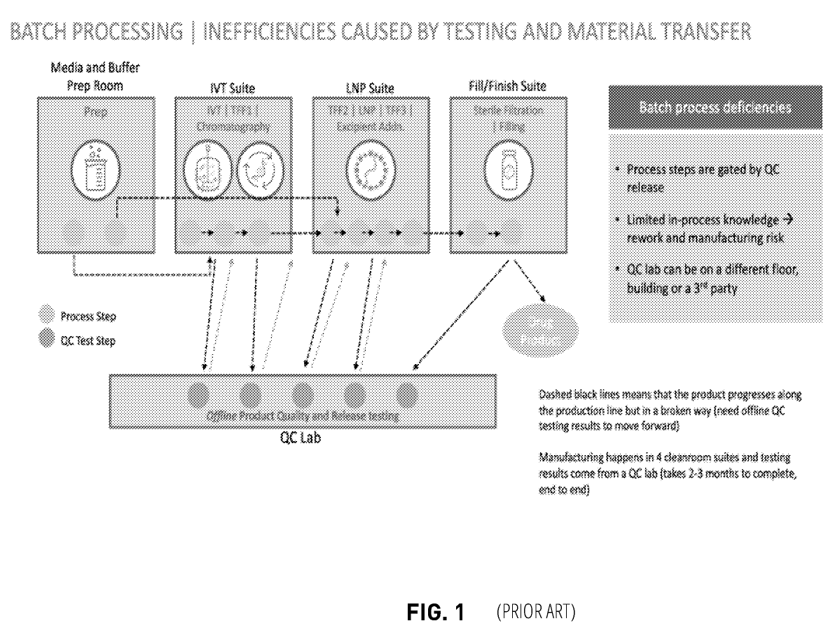

FIG. 1 illustrates a current state of the art for batch processing. At critical control points (CCPs) along the process, the samples (materials) are taken from the process and transferred to a quality control lab for off-line product quality and release testing. Such batch processing can add significant delays to the process.

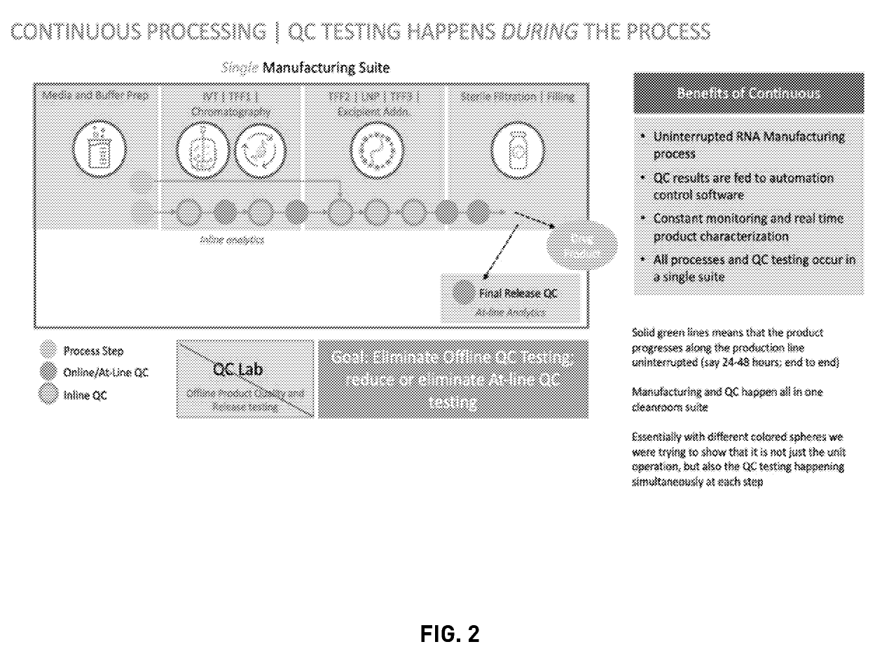

FIG. 2 illustrates a continuous processing. At critical control points (CCPs) along the process, the samples (materials) are taken in-line or at-line, processed for quality control and optimization of the process. Continuous processing may eliminate or minimize at-line processing, significantly reducing delays for quality control and may implement real-time signal processing and modelling to determine CQAs. Some real-time measurements may require data processing such as post-processing of data and modelling to obtain CQAs. Some embodiments implement multivariate modelling with mechanistic models, data-driven models, hybrid models, and machine learning models such as artificial intelligence models to obtain CQAs from data collected from in-line and, in some embodiments, at-line instruments.

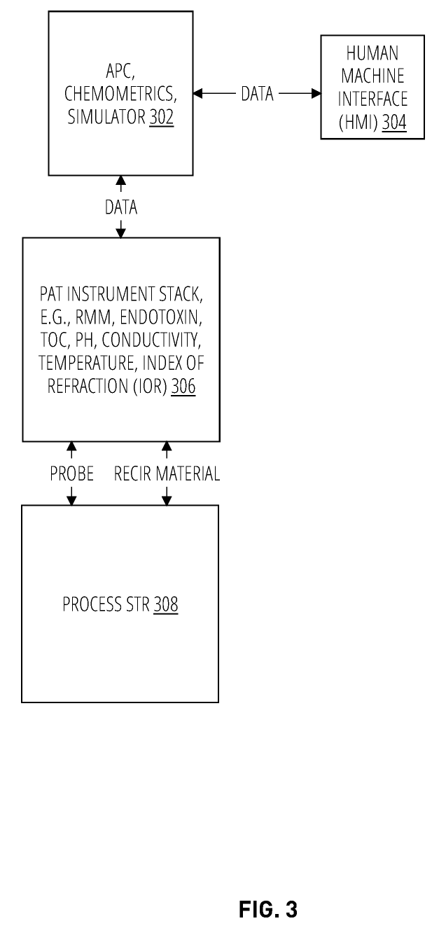

FIG. 3 is a schematic showing a mPBS and a flow of data and process material between a PAT box and a vessel, of the mPBS with a recirculation loop, or other process equipment such as the process stirred tank reactor (STR) 308, in accordance with one embodiment. In this Figure, data obtained from the PAT instruments in the PAT instrument stack 306 is analyzed by a controller, e.g., an Advanced Process Controller (APC 302) operating in conjunction with an appropriate model to determine if the product stream satisfies a predetermined quality attribute specification such as conductivity. If the specification is satisfied, the controller executes a set of instructions to release the volume of process stream to achieve RTRT utilizing predetermined models for attribute of interest.

In some embodiments, the APC 302 may perform chemometrics and simulation with a simulator model. The simulator model may perform fingerprint analysis and use data from many or all the instruments in the mPBS to assure quality control. The simulator may offer complete control over every variable, allowing analysis of the process, a profound understanding of process dynamics, and data-driven optimization for improved performance and efficiency. For instance, the simulator may simulate the entire process including measurements for each of the instruments and sensors in parallel with the process for quality control.

The simulator model may be developed based on historical data and literature about the process. In many embodiments, the simulator model for the process can be verified via offline measurements and can execute locally on the APC 302, on a local or remote server, via a web site, or on a cloud server. The simulator model may include a large model for multiple or all attributes or multiple specific attribute models and may comprise a mechanistic algorithm, a data-driven model, a hybrid model optionally with artificial intelligence (AI) and a machine learning (ML) model such as a ML statistical model, a neural network, and/or the like.

In many embodiments, the APC 302 may output data such as CQAs, trends, and simulations of the process via a human machine interface (HMI) 304. The HMI 304 may comprise a local and/or remote display or monitor, or may comprise a computer such as a laptop or workstation with a display.

In aspects, the controller may include at least four software layers. The at least four software layers include a data acquisition layer, a process scheduling layer, a deviation handling layer, and a real-time execution layer. In aspects, the software layers execute a set of programmable instructions. In aspects, the programmable instructions may be programmed in a language selected from C, Python Java, JavaScript, Perl, Tcl, or Smalltalk.

In aspects, the controller is a distributed control strategy (DCS) system including a host computer performing optimization algorithms and advanced control strategies and one or more proportional integral derivative (PID) controllers performing device level controls. The system may also include control units performing regulatory level control functions, such as PID algorithms, and may also include data gathering and extraction capabilities. The system may also include data storage devices to store process data for control and process analytics. Also included is software to communicate and interact with controllers, inputs, and outputs.

The PAT stack may include one or more instruments such as in-line instruments, at-line instrument, and offline instruments. In some embodiments, the mPBS system may include a PAT Box with 10 or more instruments such as process analytical instruments and sensors. The instruments may include, e.g., a pH meter, conductivity sensor, a total organic carbon (TOC) analyzer, a turbidity sensor, an IoR (index of refraction) system, a flow-cytometer, a light scattering particle counter, an endotoxin detector, a fluorometer, a temperature sensor, and/or a nitrate sensor. In some embodiments, an instrument may comprise a combination of two or more instruments such as a rapid bioburden instrument that may include, for instance, a combination of a flow-cytometer and a particle counter. The instruments and sensors may measure samples of water, buffer solution, CIP rinsate, drug substance, or drug product material.

In many embodiments, the instruments may access samples or perform measurements via flow cells and/or inline probes of the via a recirculation loop, a vessel of the mPBS or in-line with a process flow path of the system. The mPBS may include a vessel comprising a body defining an interior space to hold between 100ml and 100,000 liters. In aspects, the body comprises inlet and outlet ports and optionally an impeller for fluid recirculation. In aspects, the body is in the form of a flexible bag or a rigid container. In aspects, the vessel is a hold vessel, a release tank, a dilution tank, or a stirred tank reactor (STR 308).

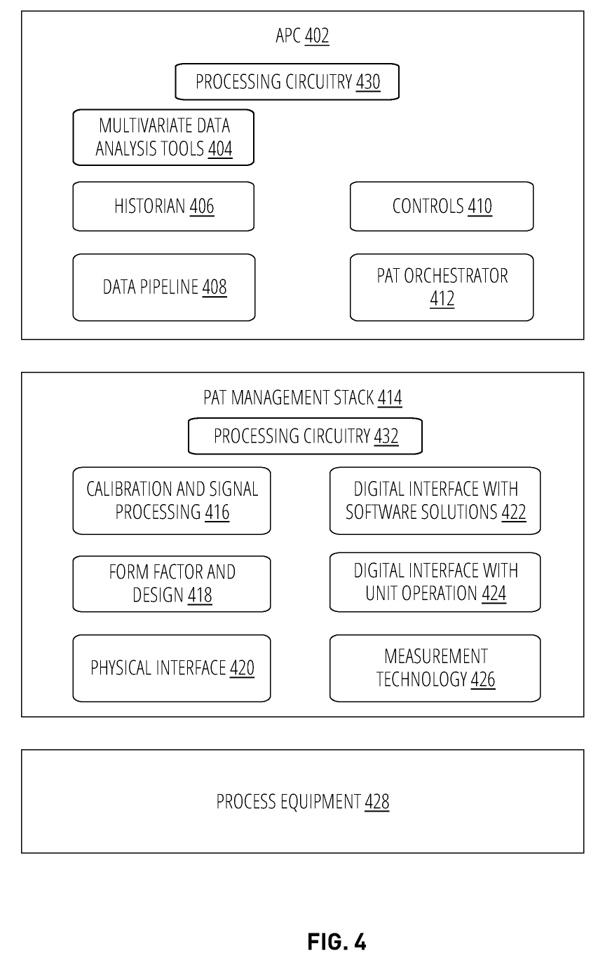

FIG. 4 schematically illustrates an embodiment of a mPBS such as the mPBS described in FIG. 3. FIG. 4 illustrates the modular framework of the mPBS including a computational/ modeling/ data analysis control system (a Communications, Data, and Analysis (CDA) stack) in an APC 402, a PAT management stack 414, as well as the integration between process equipment 428 and the PAT management stack 414.

The PAT management stack 414 may comprise processing circuitry 432 such as one or more processors coupled with memory for executing code associated with modules and may include various modules adapted to be quickly integrated in a “plug and play” fashion, each having streamlined interfaces to equipment and user systems. In other embodiments, the code associated with the various modules may execute on a separate computing platform such as the APC 402

The modules include a physical interface 420 with the process equipment 428. In aspects, the physical interface 420 is fully integrated with a single-use and/or reusable system comprising single use pre-calibrated consumables in a closed, sterile system. Other modules include scalable in-line and on-line process measurement technology 426 which measures CCPs and CQAs suitable for process control and release testing and a calibration and signal processing 416 module.

The calibration and signal processing 416 module may be pre-calibrated or auto-calibrated for one or more of the instruments and sensors. The calibration and signal processing 416 module may include no processing for some instruments or sensors, digital processing for some instruments or sensors, and calibration hardware for some instruments or sensors. For instance, an output of some instruments or sensors may be calibrated to process raw data from the instruments or sensors to be stored and/or used directly in modelling implemented by the APC 402, depending on the instrument or sensor. To illustrate, some temperature sensors may be calibrated at the manufacturer while some temperature sensors may require the digital processing of the raw data to prepare the data for later processing by the APC 402.

Some instruments may require hardware for calibration. The hardware required for calibration may depend on the specific instrument and may provide automatic calibration and recalibration, depending on the specific instrument. In some embodiments, one or more instruments or sensors may require periodic recalibration to ensure that the data collected from the instrument or sensor is accurate and precise. The hardware for calibration may include, for instance, a flow kit and/or other types of hardware. In many embodiments, the calibration and signal processing 416 module may include straightforward calibration workflows that are not disruptive to the process.

The form factor and design 418 identifies the interconnection PAT instrument stack with the process equipment 428 such as a hold tank or vessel, as well as the configuration of the physical interfaces 420 and digital interfaces with unit operations 424 to enhance modularity of the PAT management stack 414. For instance, the APC 402 may collect information or details about the instruments and/or sensors in the PAT management stack 414, the interconnection between the instruments and/or sensors and the process equipment 428, and the configuration of the physical interfaces 420 and digital interfaces with unit operations 424 to determine or select appropriate parameters for instrument operation and data collection, and models for modelling CQAs.

The digital interface with unit operation 424 may be the digital interfaces for the instruments and/or sensors of the PAT measurement stack 414. The digital interface with software solutions 422 may be the digital interfaces between instruments and/or sensors of the PAT management stack 414 and the APC 402. For instance, the digital interface with software solutions 422 may be configured to provide raw or processed data from the calibration and signal processing 416 module for various instruments and/or sensors of the PAT measurement stack 414 to the APC 402 with an organization and periodicity preconfigured and/or configured by the PAT Orchestrator 412.

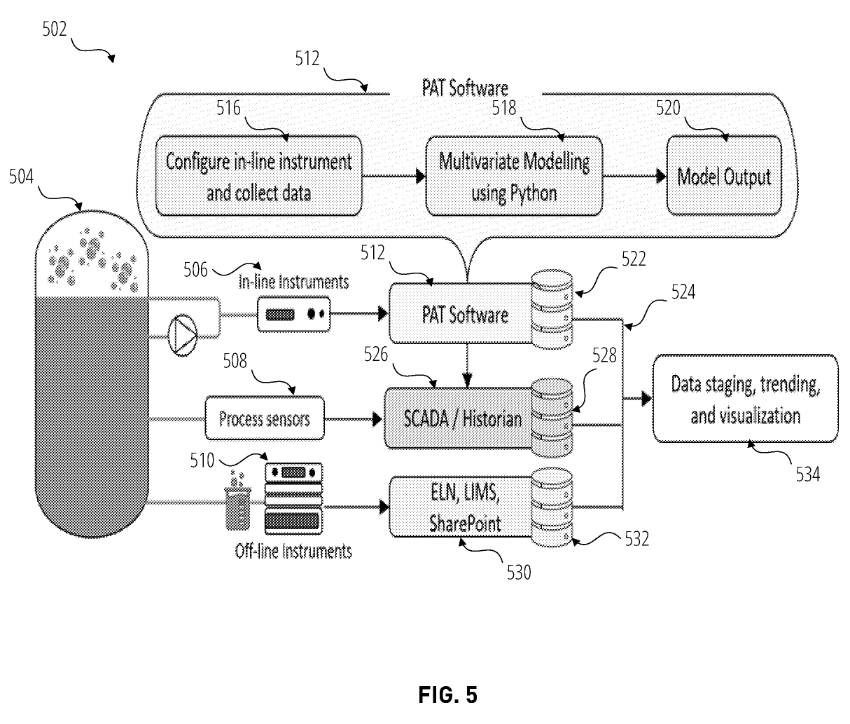

The APC 402 may comprise processing circuitry 430 such as one or more processors coupled with memory for executing code associated with modules or tools. The APC 402 may include various modules or tools including multivariate data analysis tools 404, a historian 406, a data pipeline 408, controls 410, and the PAT orchestrator 412 such as the PAT software 512 shown in FIG. 5. The multivariate data analysis tools 404 may permit real-time insight and response via accessible/static and dynamic model building, with easy-to-understand outputs having clear direction that may be integrated into process control. In many embodiments, the multivariate data analysis tools 404 may include or have access to a library of models to model each of the instruments that require modelling to determine CQAs. For instance, during an initial configuration of the APC 402, the PAT orchestrator 412 of the APC 402 may determine the configuration of the PAT management stack 414 in the process from the form factor and design 418 module. The PAT orchestrator 412 may thereafter access a remote storage or local storage comprising a library of models for modelling the instruments of the PAT management stack 414 to retrieve or load one or more appropriate models for modelling the instruments by the multivariate data analysis tools 404. In some embodiments, the PAT orchestrator 412 may also retrieve or load one or more appropriate models for simulating the process performed by the process equipment 428 that is interconnected with the PAT measurement stack 414.

The historian 406 may continuously or periodically receive and/or collect data for the process in the vessel such as the temperature, pressure, and/or the like, from one or more process sensors. The historian 406 may store the data from the process sensors 108 in a database or other data structure for use in data staging, trending, and visualization. In some embodiments, the historian 406 may include a supervisory control and data acquisition (SCADA) system.

The data pipeline 408 may be a physical data interface for communication with and control of the PAT management stack 414 by the APC 402. The controls 410 module may include an instrument control module for commanding data acquisition and/or collecting measurement data from each of the instruments of the PAT management stack 414. For some instruments, e.g., the measurement data may comprise an array or spectra of raw data per measurement.

The APC 402 may also comprise the PAT orchestrator 226, which provides automated data collection, extraction, harmonization, and storage from measurement tools that enable design of experiments (DoE), multivariate data analysis (MVDA), technology transfer, and batch record integration.

FIG. 5 illustrates a system 502 of a process with in-line instruments 506, process sensors 508, and off-line instruments 510. The system 502 may also comprise a PAT management and operations system with PAT software 512 comprising a configure in-line instrument 506 and collect data module 516, a multivariate modelling module 518, and a model output module 520. The PAT management and operations system may manage, configure, and collect measurement data from the in-line instruments 506, input the measurement data into a model such as a multivariate model, generate a model output including real-time transformed data and trending for CQAs, and output the model output via display or other device to present the real-time transformed data and trending in, e.g., a plot or other visual output, for the CQAs.

The configure in-line instrument and collect data module 516 may interact with a user to establish one or more parameters for collection of raw measurement data from the in-line instruments 506. For instance, the configure in-line instrument and collect data module 516 may command acquisition parameters for the in-line instruments 506 to establish, e.g., a periodicity or other timing for acquisition of measurement data from the in-line instruments 506 and collect the data from the in-line instruments 506. Establishing the one or more parameters for collecting measurement data from the in-line instruments 506 may comprise establishing the one or more parameters specific to a manufacturing process and specific to each unit operation for the in-line instruments 506 in that manufacturing process. In many embodiments, the PAT software 512 may determine the manufacturing process, such as a Water for injection (WFI) process, and the unit operation for the in-line instruments 506 from a form and factor design module such as the form factor and design 418 module shown in FIG. 4.

The in-line instruments 506 may couple with a vessel 504 of the specific manufacturing process for the mPBS and have a specific unit operation in that manufacturing process. For example, at least one of the in-line instruments 506 may comprise a Rapid Bioburden instrument and a specific unit operation for the Rapid Bioburden instrument such as chromatography using an OligodT monolith column measured by a rapid microbial method (RMM) system to determine cell count of viable bioburden cells. As another example, the in-line instruments 506 may comprise conductivity, TOC (Total Organic Carbon), pH, temperature, index of refraction and bioburden in real-time and the unit operation may be a Tangential Flow Filtration (TFF), or Cross-flow filtration. The mPBS may be installed between each unit operation, whether that be between a bioreactor and a centrifuge, a chromatography skid and TFF system or any two pieces of bioprocessing equipment that are known by those skilled in the art.

The multivariate modelling module 518 may receive the measurement data from the configure in-line instrument and collect data module 516 and run, e.g., Python codes or other codes to process raw measurement data or processed data and execute a model that is specific to a unit operation for a specific process for manufacturing , e.g., WFI. In some embodiments the Python codes or other codes may execute locally and, in other embodiments, the Python codes or other codes may execute in a cloud server via a model execution web app. The model execution app may execute Python models that can handle real-time data execution to output real-time results. In some aspects, the Python models may comprise pre-treatment methods including interpolating functions, derivation, and normalization; machine learning models such as multivariate models including principal component analysis (PCA) and partial least square (PLS) regression; and hybrid models by combining mechanistic equations and multivariate models.

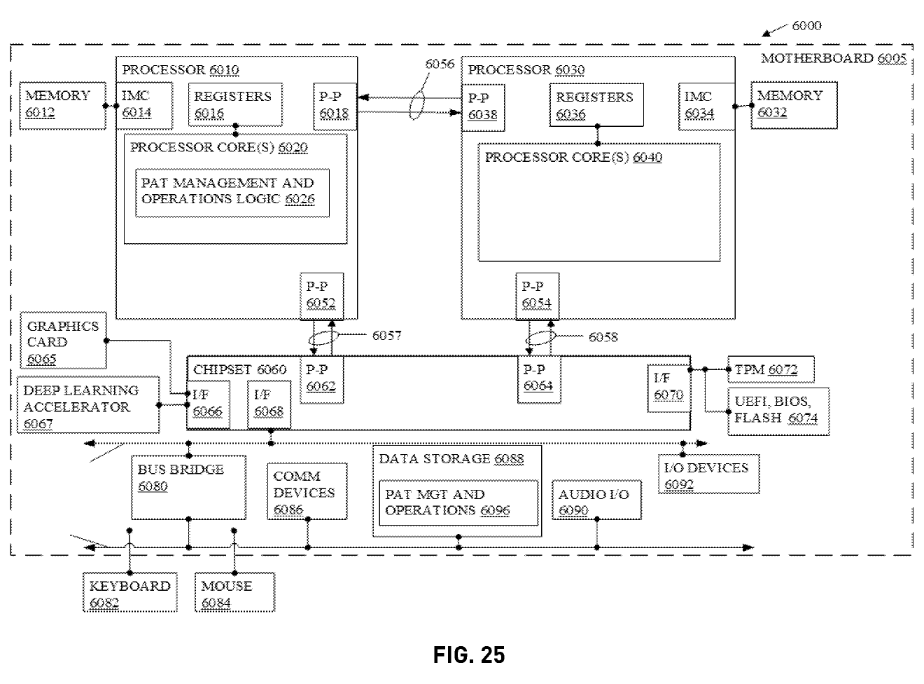

The multivariate modelling module 518 may perform measurement sequencing that includes acquiring measurement data, monitoring measurements status, raising errors if measurements fail, storing measurement data, transferring measurement data to a local model execution app or web app, receiving transformed data and model outputs from the model execution app or web app, and storing transformed data and model outputs in a local or remote data storage 522. In some aspects, the local or remote data storage 522 may comprise a centralized data storage for the PAT software 512. In some embodiments, the centralized data storage may comprise a centralized data server such as the computer 6000 shown in FIG. 25, which is accessible via a programming language such as a structured query language (SQL). In many embodiments, the raw or processed measurement data, transformed data, and trending data in the data storage 522 are available to a data staging, trending, and visualization system 524 for further analytics and visualization.

The multivariate modelling module 518 may pass the transformed data and the model output to a model output module 520. The model output may include the transformed data such as a real-time CQA and trending data for the CQA. The model of the multivariate modelling module 518 may determine or predict the trending data based on a library of trending data for the CQA for the specific unit operation in the specific process for manufacturing. In many embodiments, the CQA may include a volume, a concentration, or a mass.

The model output module 520 may display the real-time transformed data and trending data on a display, on a plot, or on another visual output locally and/or remotely in real-time.

In some embodiments, the PAT software 512 may enable two-way communication between any PAT, or in-line instrument 506, that is capable of transferring data using Open Platform Communications Unified Architecture (OPC-UA) and Representational State Transfer Application Programming Interface (RestAPI). In some aspects, the PAT software 512 may enable concurrent operation of multiple in-line instruments 506 and models in real-time.

The process sensors 508 may continuously or periodically collect data for the process in the vessel such as the temperature, pressure, and/or the like. The data collected by the process sensors 508 may be sent to a supervisory control and data acquisition (SCADA)/historian system 526. The SCADA/historian system 526 may store the data from the process sensors 508 in a database or other data structure for use by a data staging, trending, and visualization system 524.

The off-line instruments 510 may collect samples for off-line analysis and may send the data for the samples to an electronic notebook (ELN), a laboratory information management system (LIMS), and/or SharePoint system 530 for storage in database or other data structure. In aspects, the data captured by the ELN, LIMS, and/or SharePoint system is sent to the data staging, trending, and visualization system 524.

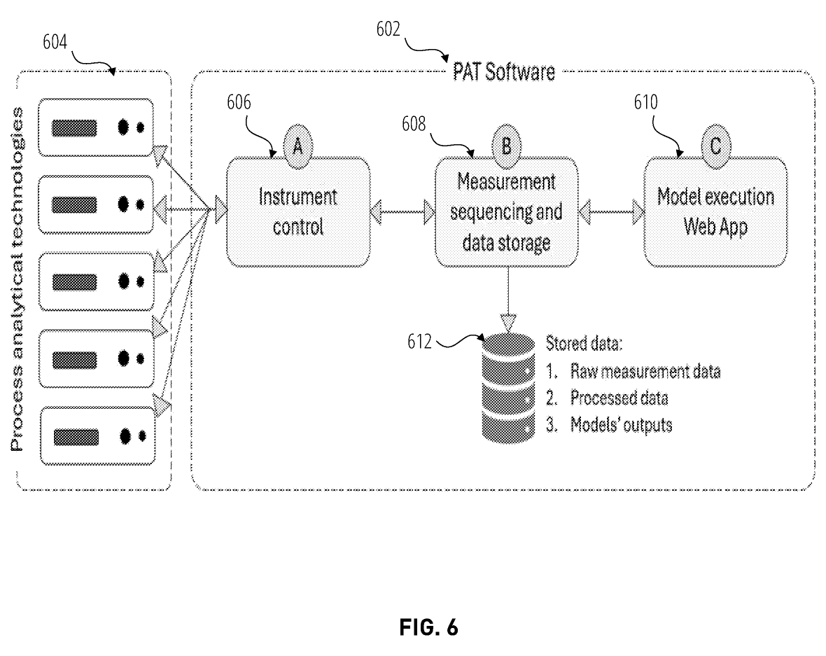

FIG. 6 illustrates another system of a process with in-line instruments (or PATs) 604 and a PAT software 602 (such as the PAT software 512 shown in FIG. 5, with data flow indications and model execution in a web server. The PAT software 602 may include an instrument control module 606 such as the configure in-line instrument and collect data module 516 shown in FIG. 5, a measurement sequencing and data storage module 608, and a model execution web app 610. The instrument control module 606 may configure, manage, and collect measurement data from the in-line instruments 604 where the configuration flows from the instrument control module 606 to the in-line instruments 604 and the raw measurement data or processed measurement data flows from the in-line instruments 604 to the instrument control module 606. The instrument control module 606 may also manage measurement data collection by receiving an instrument status from the in-line instruments 604 including instrument ready, instrument offline, and instrument configured. In some embodiments, the instrument control module 606 may manage the starting and stopping of collection of raw measurement data from one or more of the in-line instruments 604.

The instrument control module 606 passes the measurement data from the in-line instruments 604 to the measurement sequencing and data storage module 608. The measurement sequencing and data storage module 608 may acquire measurement data; monitor measurement status; raise errors to, e.g., user via display or remotely messaging, if measurements fail; store raw or processed measurement data in a PAT database 612 of a data storage device; and transfer measurement data to the model execution web app 610 for processing.

The model execution web app 610 may receive the raw or processed measurement data for each of the in-line instruments 604 and enable execution of models such as Python models in a Python code concurrently, in real-time, to process the measurement data. Examples of the processing of measurement data include: Pre-treatment methods (interpolating functions, derivation, and normalization), Multivariate models including principal component analysis (PCA) and partial least square (PLS) regression, and Hybrid models by combining mechanistic equations and multivariate models.

The model execution web app 610 may send transformed data and the model output to the measurement sequencing and data storage module 608. The transformed data may include CQAs and the model output may include trending data identified based on predictions, estimations, or calculations of the models based on the measurement data and a library of trending data for the CQAs. Then, the measurement sequencing and data storage module 608 may store raw or processed measurement data, transformed data, and models’ outputs in the data storage module 612 such as a central data server using, e.g., SQL. In the central data server, the data is readily available for external data analytics platforms.

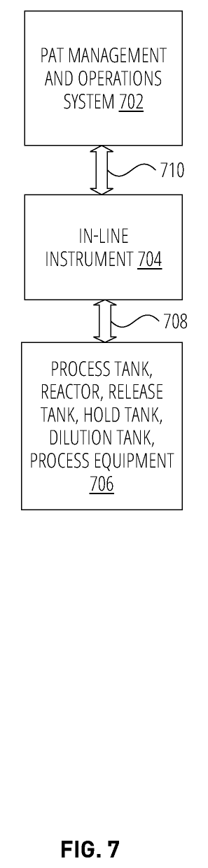

FIG. 7 schematically illustrates an embodiment of a mPBS with a PAT management and operations system 702 coupled with an in-line instrument 704 of the mPBS and a vessel 706 of the mPBS as described herein. The PAT management and operations system 702 may communicatively connect to the in-line instrument 704 to configure, manage, and collect data from the in-line instrument 704. The in-line instrument 704 may include one or more analytic instruments with associated flow cells and probes. Exemplary flow cells that may be included are conductivity, TOC (Total Organic Carbon), pH, temperature, index of refraction (IoR), and bioburden flow cells. As illustrated, the in-line instrument 704 is in fluid communication with a unit operation of a WFI manufacturing process, either directly or via an intervening vessel 706 coupled with the in-line instrument 704. The vessel 706 may be, for example, a process tank, a reactor, a release tank, a hold tank, a dilution tank, or a STR.

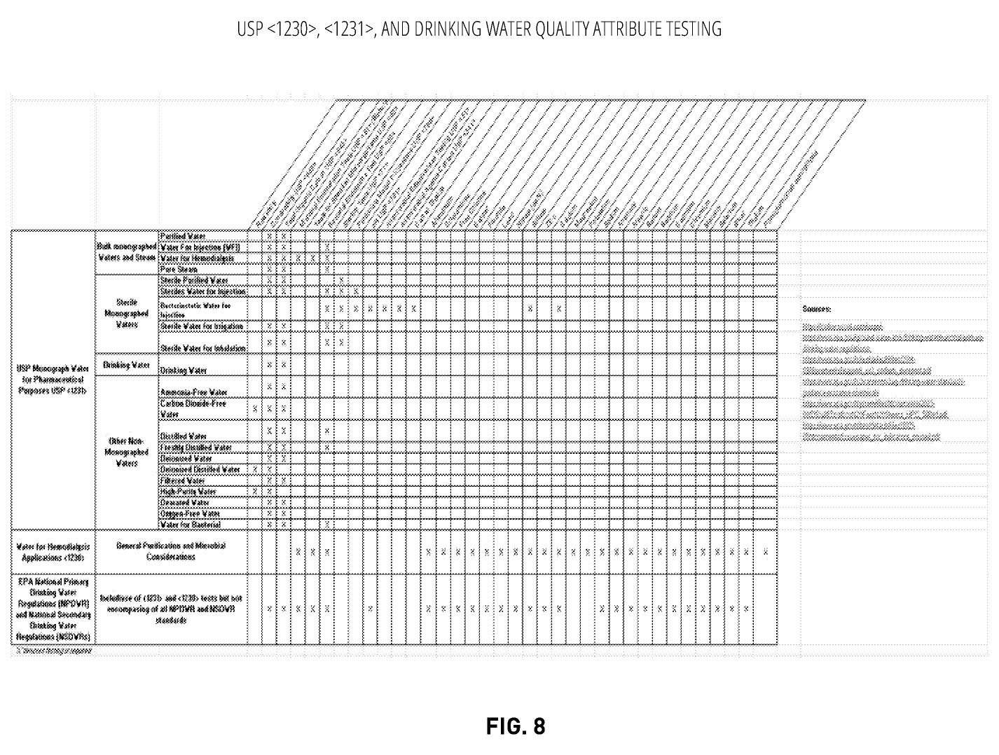

FIG. 8 is a testing matrix showing the required tests for USP monographs <1230> and <1231> as well as tests from the monographs that overlap with testing required for drinking water per the EPA. This figure illustrates the wide range of applications of purified water and how the various test method requirements change between applications. For example, the minimum testing required for Water for Injection (WFI) in most biopharmaceutical manufacturing applications is conductivity, TOC, bioburden, and endotoxin. However, the minimum testing required for Bacteriostatic Water for Injection is endotoxin, sterility, particulate matter, pH, antimicrobial agents, carbon dioxide, sulfate, and calcium. Overall, the testing requirements that remain the most common are conductivity, TOC, endotoxin, and bioburden or sterility.

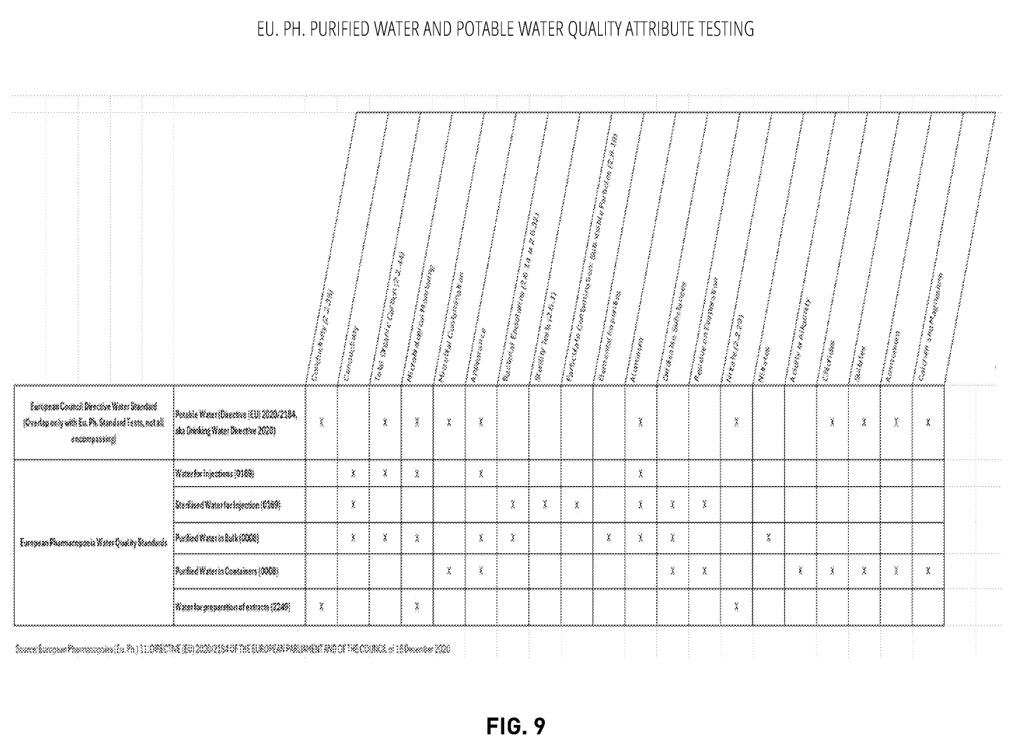

FIG. 9 is a test matrix showing the required tests for Eu. Ph. Monographs 0169, 0008, and 2249 as well as tests from the monographs that overlap with testing required for potable water per the European Council Directive. Like the USP testing requirements, there are several different applications for purified water according to the Eu. Ph. such as Water for Injections and Purified Water in Bulk. These various water applications require several different test methods as well with the most common tests being conductivity, TOC, bioburden or sterility, and aluminum.

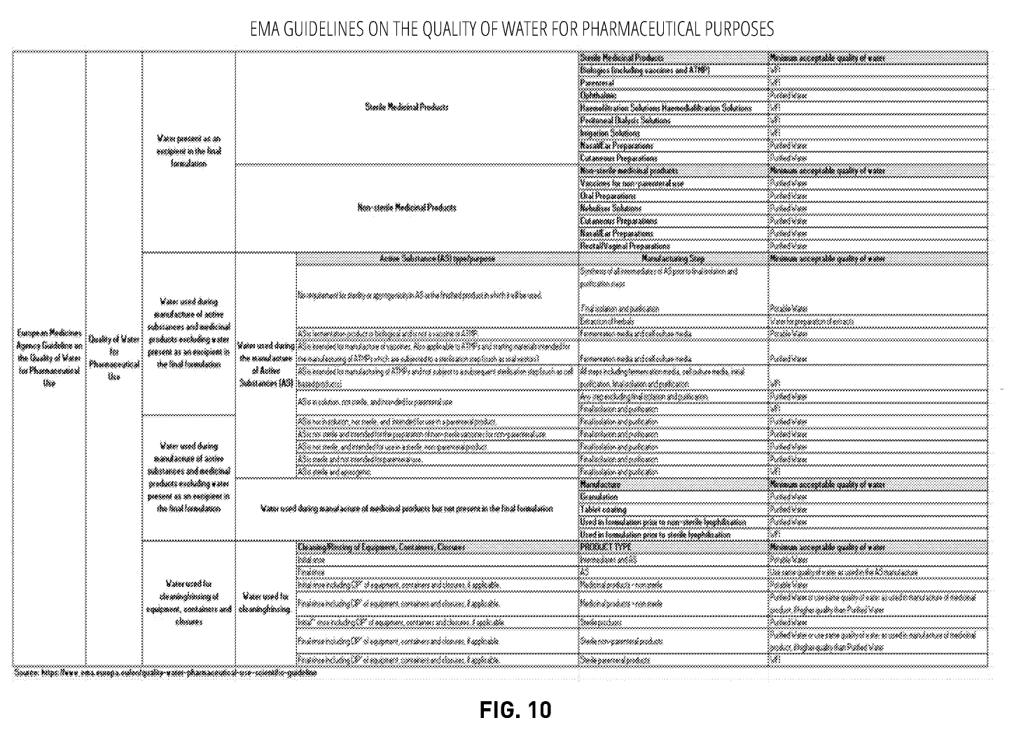

FIG. 10 is a tabulated summary of the EMA Guidelines on the Quality of Water for Pharmaceutical Use which specifies either Purified Water, WFI, or Water for preparation of extracts for various applications. This figure gives a more expansive look at the various applications of different purified water types and potable water throughout various pharmaceutical use cases according to the EMA guidance. This figure further evidences the broad use of purified water across the landscape of multiple pharmaceutical applications and implicitly illustrates the multitude of testing that is required.

Purified water is a raw material used daily in most pharmaceutical development and manufacturing facilities and makes up a significant portion of all samples tested in pharmaceutical facilities. FIG. 8 and FIG. 9 show commonality of tests required for purified water and potable water. FIG. 10 shows the abundance of applications of purified water. Purified water generation and distribution systems typically run 24 hours/day and 365 days/year in most facilities. Upkeep of these systems is expensive, and downtime of these systems can be very costly as it prevents manufacturing from moving forward causing fewer products to be produced each year as well as fewer customers being supported by contract manufacturing organizations. Faster turnaround of these water quality tests could improve maintenance of these purified water generation systems and decrease downtime. Moving more towards in-line and on-line methods for testing these water quality attributes would release purified water and systems closer to real time, and significantly decrease the cost of routine off-line methods and improve overall productivity.

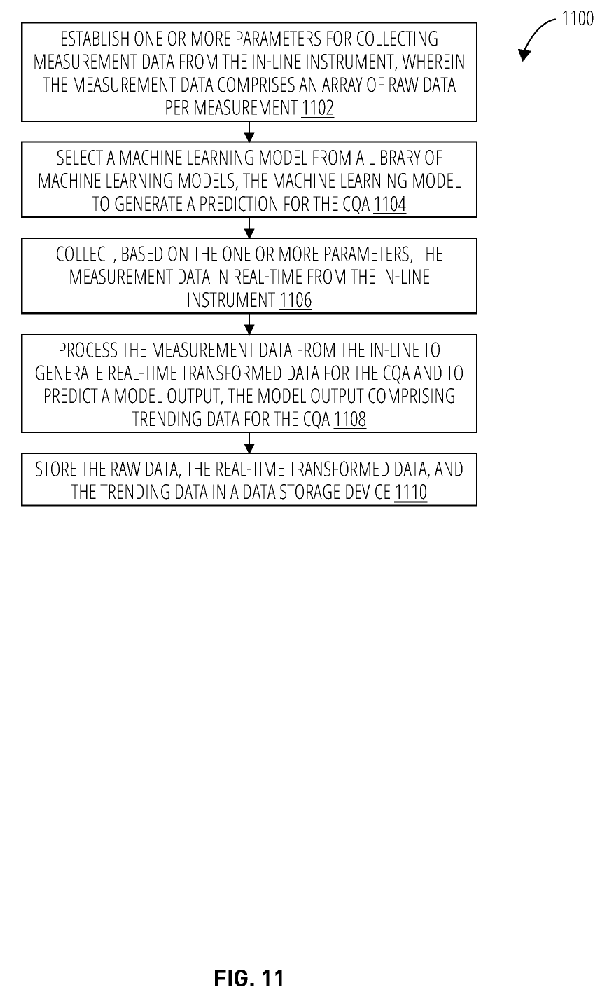

FIG. 11 illustrates a flowchart for a process 1100 for a PAT management and operations system such as the PAT management and operations system 702 shown in FIG. 7 to establish monitoring for a Critical Quality Attribute (CQA) in real-time for an in-line instrument such as the in-line instrument 704 shown in FIG. 7. In block 1102, process 1100 establishes one or more parameters for collecting measurement data from the in-line instrument, wherein the measurement data comprises an array of raw data per measurement. In some embodiments, establishing one or more parameters for collecting measurement data from the in-line instrument may comprise establishing the one or more parameters specific to a manufacturing process and specific to each unit operation for the in-line instrument in that manufacturing process. In some embodiments, the PAT management and operations system may remotely control collection of raw measurement data from the in-line instrument such as starting collection of measurement data, stopping collection of measurement data, configuring parameters for collection of measurement data, and tracking an in-line instrument status for collection of measurement data such as ready, offline, configured, and/or the like.

In some embodiments, establishing one or more parameters may comprise commanding acquisition parameters or measurement configurations for the in-line instrument. In some aspects, the CQA for an in-line instrument comprises a volume, a concentration, or a mass.

The in-line instrument may include, for example, rapid microbial method (RMM) analyzer, endotoxin detector, a Total Organic Carbon (TOC) analyzer, a pH meter, conductivity sensor, temperature sensor, and an index of refraction (IoR) system.

In block 1104, process 1100 selects a machine learning model from a library of machine learning models, the machine learning model may then transform the raw measurement data into a real-time CQA and may generate a prediction for the CQA such as trending data based on a library of trending data for the CQA. In some aspects, selection of the machine learning model may be based on the specific manufacturing process and may be specific to a unit operation for the in-line instrument in that manufacturing process.

In some aspects, the library of machine learning models comprises models for a set of specific unit operations for a process for WFI generation. In some aspects, the set of specific unit operations comprise one of a bioreactor, a centrifuge, a chromatography skid, a TFF system or any bioprocessing equipment that are known by those skilled in the art.

In block 1106, process 1100 collects, based on the one or more parameters, the raw measurement data in real-time from the in-line instrument. For instance, the one or more parameters may identify a periodicity for collection of the measurement data such as every 5 minutes, every 3 minutes, every 2 minutes, every minute, or the like.

In block 1108, process 1100 processes the raw measurement data from the in-line to generate real-time transformed data for the CQA and to predict a model output. The model output may determine trending data for the CQA by, e.g., predicting the trending data for the CQA based on a library comprising multiple sets of trending data for the CQA. In some aspects, processing the raw measurement data may comprise enabling execution of Python models in a model execution web app or a locally executable model execution app. For example, the model execution may reside in a cloud server or a local server such as the computer 6000 shown in FIG. 25. The model execution web app may receive the raw measurement data from measurement sequencing and run or execute Python codes to process raw measurement data.

In some aspects, the model execution web app executes any Python models that can handle real-time data. The Python models may comprise pre-treatment methods including interpolating functions, derivation, and normalization; multivariate models including principal component analysis (PCA) and partial least square (PLS) regression; and hybrid models by combining mechanistic equations and multivariate models.

In block 1110, process 1100 stores the predicted trending data in a data storage device. The machine learning model may also store the raw measurement data and the transformed data (CQA) in a database or other data structure so that the raw measurement data and transformed data (or processed data) are available for visualization software and/or analytics software. In some aspects, storing the trending data in a data storage device comprises measurement sequencing, wherein measurement sequencing comprises storing the raw measurement data, the real-time transformed data, and the real-time trending data in a centralized database of PAT measurements and machine learning model outputs. In some embodiments, the data storage device comprises a centralized database that is accessible via a structured query language (SQL).

In some embodiments, the process 1100 may further comprise displaying via, e.g., a HMI, the real-time transformed data and real-time trending data. In further embodiments, the process 1100 may enable two-way communication between any in-line instrument that is capable of transferring data using Open Platform Communications Unified Architecture (OPC-UA) and Representational State Transfer Application Programming Interface (RestAPI).

According to some aspects, the raw data, the real-time transformed data, the trending data, and the machine learning models are auditable. For instance, the process 1100 may maintain audit trails, version control, and security such as user authentication and user groups.

In further embodiments, the process 1100 may further comprise enabling concurrent operation of multiple instruments and multiple models in real-time.

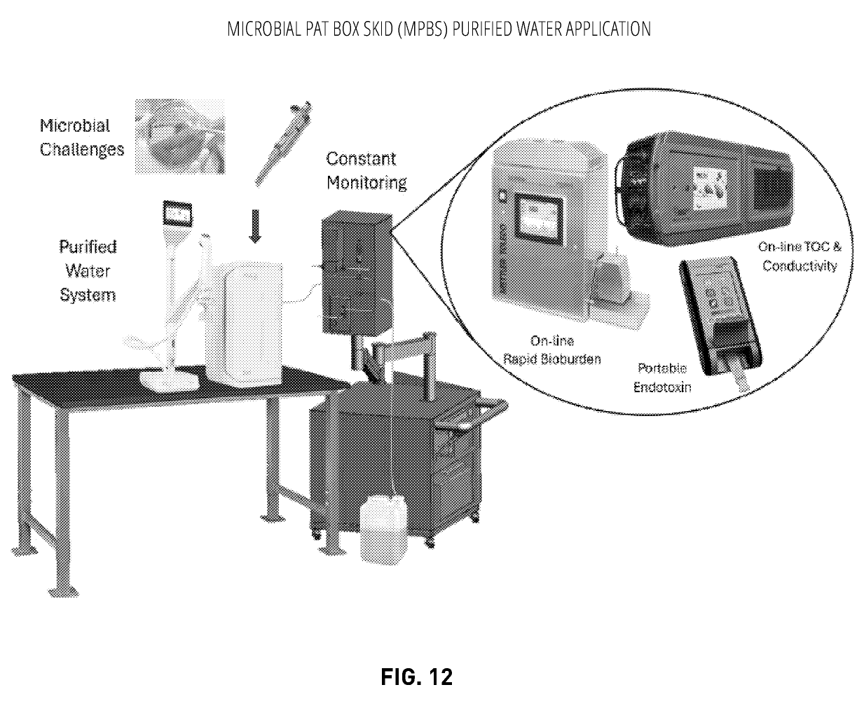

FIG. 12 is an example of a custom designed mPBS that fully integrates the analytical technologies from on-line rapid bioburden, on-line TOC, in-line conductivity, and at-line endotoxin into a PAT Box connected to and monitoring a purified water system. This mPBS fully encloses the base instrumentation, transmitters, and or industrial PCs and control systems needed to operate all the PAT instruments in concert while sampling purified water process and result analyses. In many embodiments, the PAT box may comprise on-line flow cells and a vial or other vessel to allow flow of fluid or material through on-line flow cells and to sample small aliquots of the fluid or material for at-line testing such as endotoxin detection.

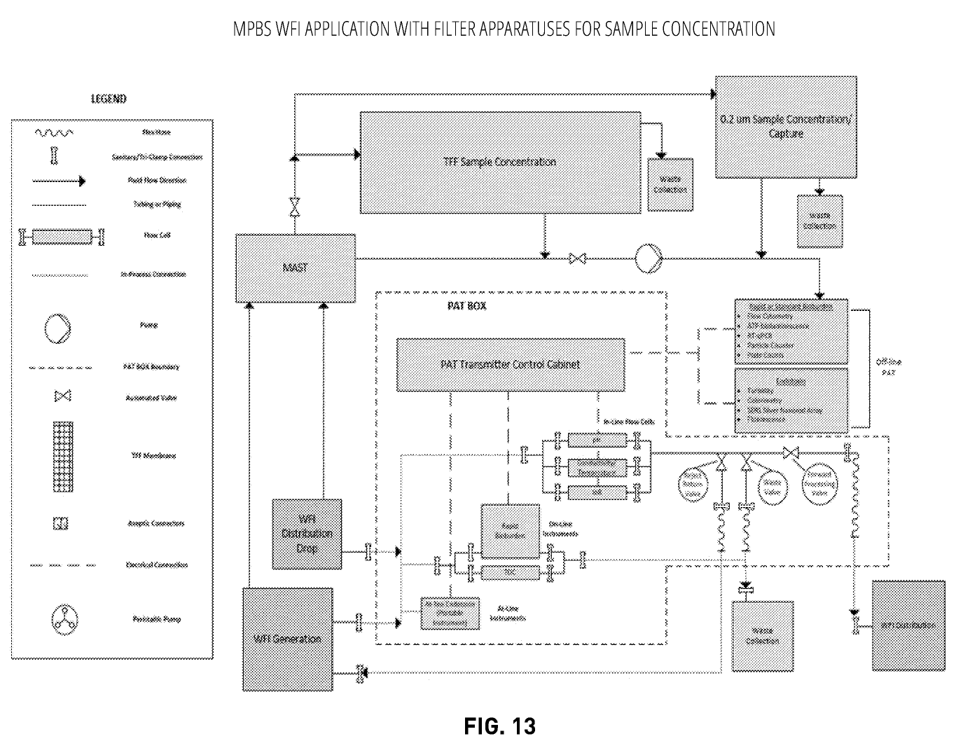

FIG. 13 is a schematic diagram showing the mPBS situated downstream of a WFI generation skid and/or WFI distribution drop. The diagram also shows how automated sampling of a WFI generation and distribution drop can be routed through an automated sampling system, such as a MAST, to off-line PAT directly or through various sample filtration or concentration apparatuses. In some embodiments, the mPBS is connected to a filtration system capable of concentrating samples.

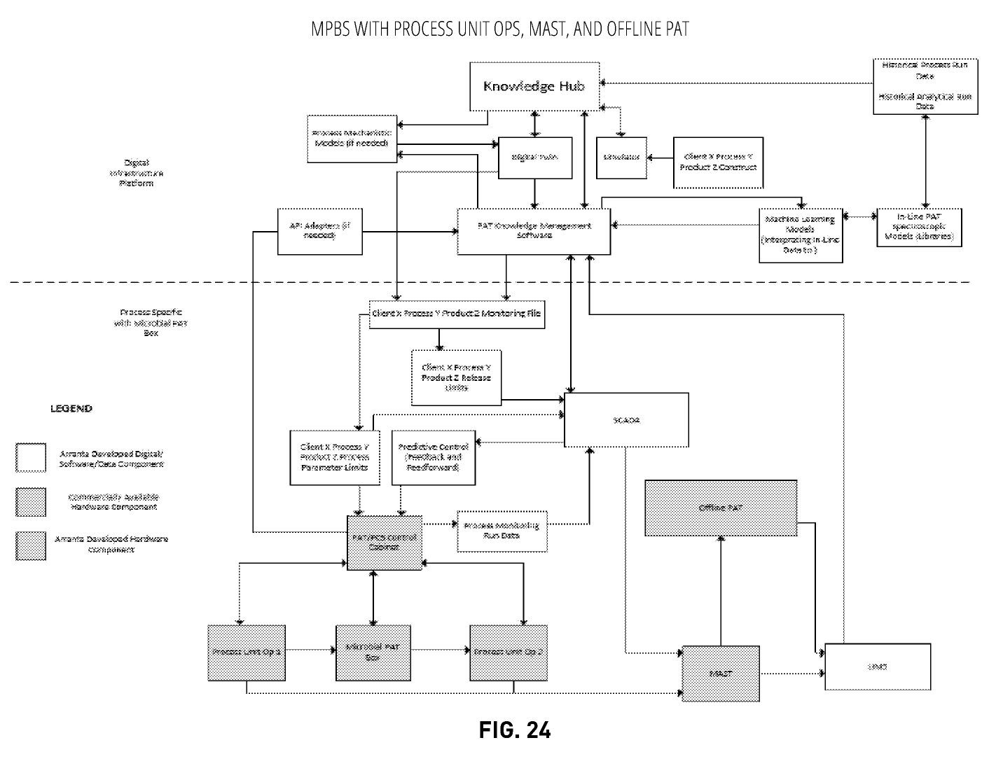

FIG. 13 is a schematic diagram illustrating the specific in-line and off-line PAT as well as two filtration methods used for sample preparation for off-line PAT methods. The mPBS may be installed permanently to a WFI generation mPBS or operate as a mobile cart which can be moved around a facility and sample multiple WFI generation and/or distribution drops throughout the facility. The mPBS may use in-line flow cells to monitor attributes such as conductivity, TOC (Total Organic Carbon), pH, temperature, index of refraction, and bioburden in real-time. The mPBS may sample WFI drops and/or generation sample points and transfer the sample to off-line PAT to measure endotoxin and bioburden. An example of a commercially available on-line bioburden PAT system may be the flow cytometry, particle counter, and/or fluorometer. Examples of in-line and off-line PAT are shown but are not intended to be limiting. Modular Automated Sampling Technology (MAST) may also pull WFI generation samples and transfer them to a TFF sample concentration station or a depth filtration station that might be a 0.2 um filter. The TFF concentration station works in a manner typical of TFF where a known analyte is concentrated so that an off-line PAT instrument may be able to detect the analyte. In a similar fashion, an analyte may be processed and captured by a depth filter where the analyte is then backflushed off the filter and into a receptible and then transferred to off-line PAT for analysis.
1. PAT data is sent to the PAT Control Cabinet and then sent to the PAT Knowledge Management software and WFI monitoring file (as shown in FIG. 24) for processing. The SCADA will inform the PAT control cabinet whether the WFI generation samples meet criteria and may pass to WFI distribution or pass the criteria in general in relation to the WFI drop samples. If they do not pass the criteria, the WFI may be recycled back to the WFI generation system for re-processing or may be transferred to waste.
2. The PAT instruments may include one or more analytical instruments for determining one or more attribute measurements for WFI. The PAT instruments may include a refractometer for measuring refractive index (IoR), and/or a bioburden analyzer, conductivity and TOC sensors. Additionally, such instruments can also include nitrate sensors as well as pH and temperature sensors.

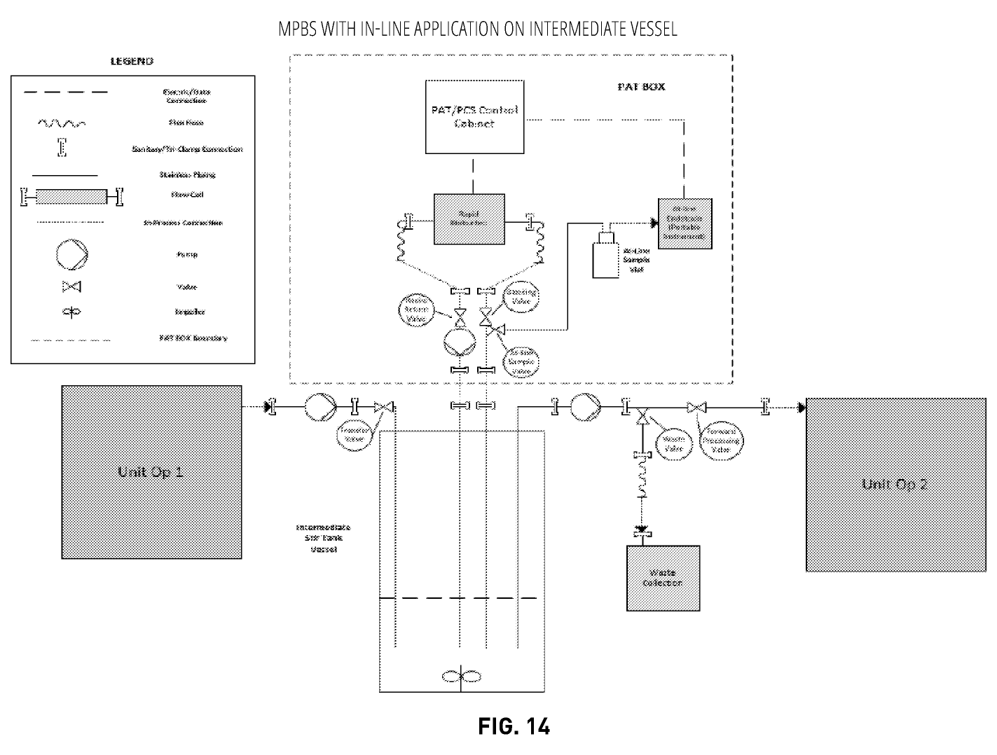

FIG. 14 is a schematic diagram illustrating a process flow diagram (PFD) of mPBS where the RMM system is situated in-line, and the endotoxin instrument is situated in an at-line configuration. The PAT Box boundary is shown by dashed lines and FIG. 14 shows the PAT Box of the mPBS situated where the sample is drawn from an intermediate process vessel of the mPBS that sits between drug manufacturing process system unit operations. The recirculation loop also has a side stream connected for at-line sampling. FIG. 14 includes the RMM system with an in-line configuration. The intermediate vessel can be sampled into a sample container for transfer to the at-line endotoxin systems and/or recirculate material from the vessel through RMM system sample port to facilitate direct sampling if the in-process material is dilute enough to be measured by the RMM system. The RMM system and the endotoxin system are connected to the PAT/process control system (PCS) control cabinet which sends commands and receives result data from the RMM and endotoxin instruments. Result data can then be used to command process material via smart valves to flow from the vessel to either Unit Op 2, waste collection, or back to a previous unit operation depending on where results fall within certain criteria.

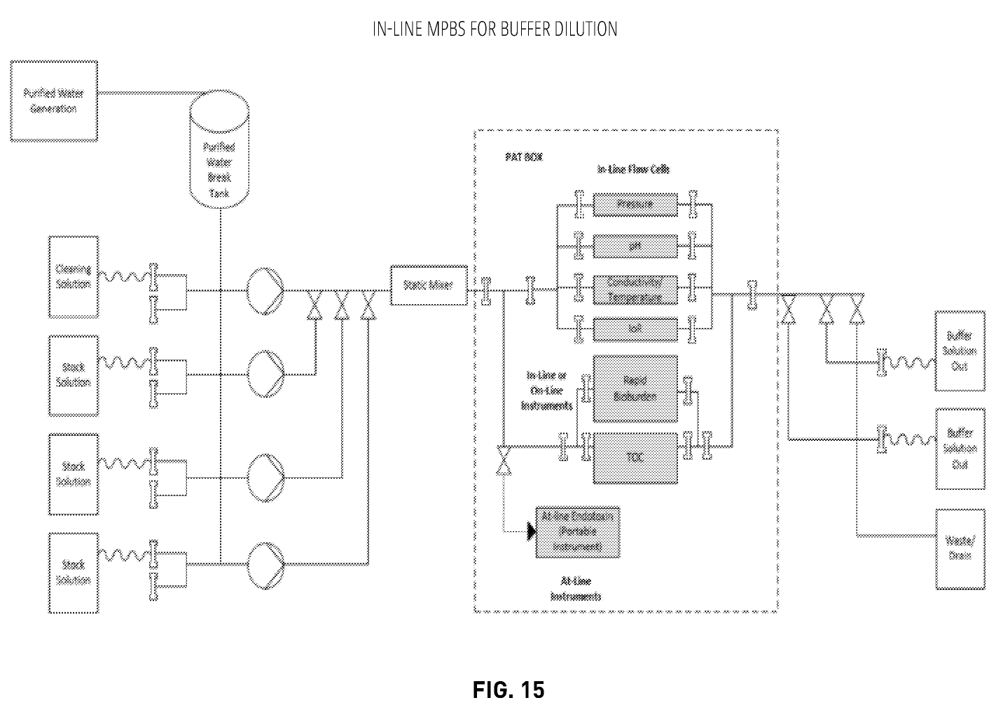

FIG. 15 is a schematic showing the mPBS connected to the outlet manifold of a buffer dilution skid. A buffer dilution skid works to mix buffer stock concentration with purified water to produce a larger volume of diluted buffer and collect the diluted buffer in a container or vessel for transfer to other parts of a drug or reagent manufacturing process. Buffers typically need testing to ensure quality before being used in further processes. Because of the low cost and larger volumes of buffers typically produced, the RMM and TOC system may be placed in-line. Additionally, a pressure sensor, pH sensor, conductivity/temperature sensor, and IoR flow cells are also configured in-line. An at-line side stream is shown for sample collection for the at-line endotoxin instrument. In many embodiments, the mPBS may comprise a vessel such as a vial to hold the sample collected for at-line endotoxin detection.

The PAT Box of the mPBS is placed in-line between the static mixing unit and the buffer dilution skid outlets connected to the final collection containers to enable real time measurement of CQAs of the buffers prior to filling in their containers.

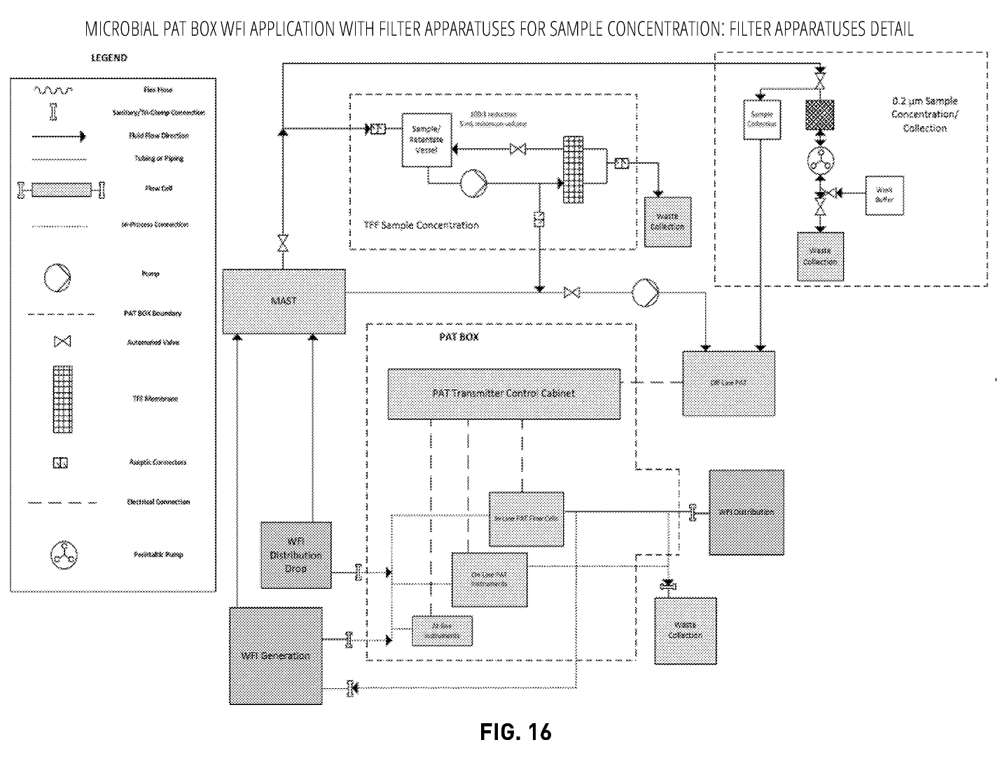

FIG. 16 is a schematic diagram illustrating a PFD like FIG. 13, however showing less detail on the Microbial PAT Box and greater detail on the inner workings of the TFF Sample Concentration apparatus and the 0.2 um sample concentration and collection apparatus.

3. The TFF Sample Concentration section comprises a typical TFF recirculation loop inline with a retentate vessel, feed pump, filter membrane and retentate back pressure valve. Once the retentate volume is concentrated to a certain predetermined point, the entire TFF tubing section can be drained at a low point and send the bulk to the MAST system for collection.

4. The 0.2 µm filter sample concentration section works to send the initial sample through the 0.2 µm filter. Once all the initial sample is sent through the filter, the retained analyte can be backflushed off the front of the filter and be collected separately for transfer to off-line PAT.

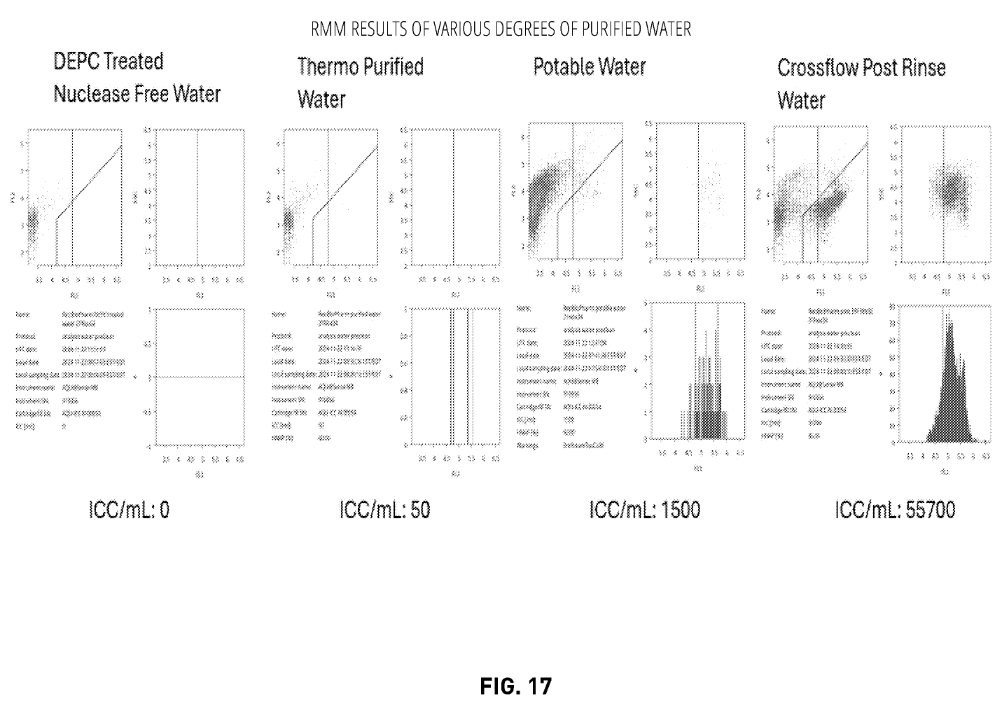

FIG. 17 shows results of several samples of water ranging from ultra purified to water that is likely contaminated with microbial material. These are results of tests performed with a rapid microbial method (RMM) equipment system illustrating the potential of these RMM systems in biopharmaceutical settings. The left-hand side of the gate on each result’s dot plot signifies non-viable cells and the right-hand gate signifies viable cells. The diagonal line signifies the separation where non-viable cells are counted above the diagonal line and viable cells are counted below the diagonal line. Using this gating scheme, one can ascertain the trend of increasing non-viable and viable cell counts starting from the most purified water sample (DEPC Treated Nuclease Free Water) on the far left of the figure and progressing right to Purified Water (via a Thermo branded purified water system), potable water (sampled from a municipal water source), and water sampled from water flushed through an uncleaned TFF lab system. As seen in the figure, the ICC (cell count) increases as the purification level of the water decreases, showing a logical trend. Most notably, this method can be used quickly identify the level of cleanliness of biomanufacturing systems such as TFF.

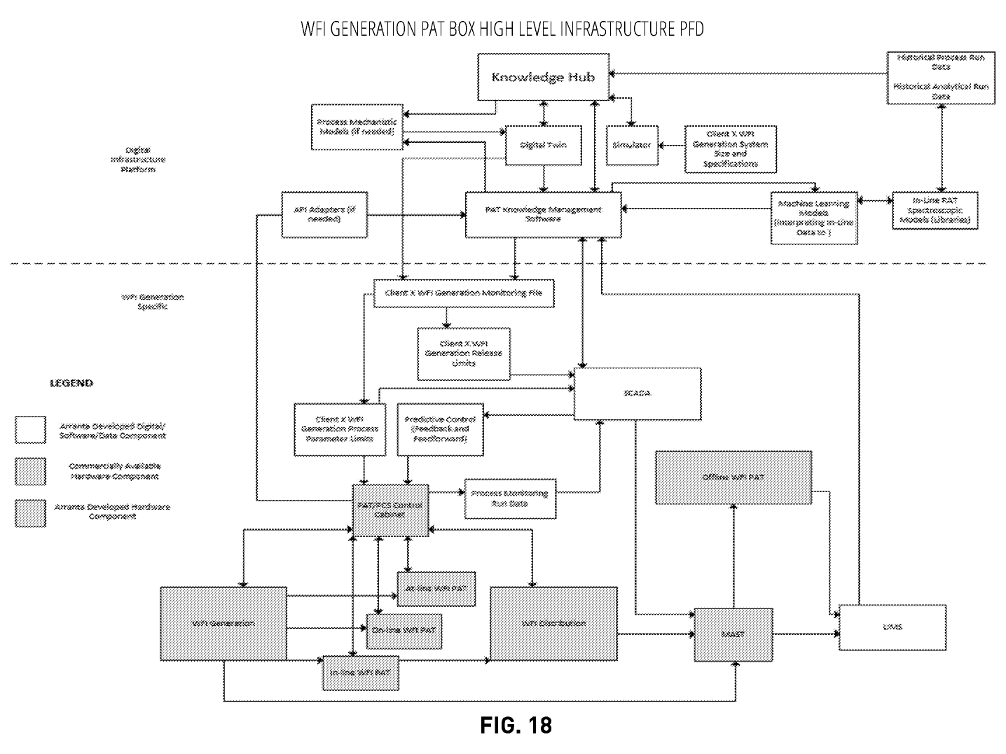

FIG. 18 is a schematic diagram illustrating a PFD of an aspect of the Microbial PAT Box ecosystem specific to controlling and monitoring water for injection (WFI) generation and distribution with the Microbial PAT Box connected to a WFI generation system. The diagram also shows the interconnectedness of the Microbial PAT Box between the WFI Generation system and the digital infrastructure which drives PAT results analysis and feedback control loops through the Microbial PAT Box and into the WFI generation system.

5. The PAT instruments sample the WFI generation system and feed results data through the PAT/PCS control cabinet in the form of process monitoring data through the SCADA platform to the PAT Software and the results are historized and sectioned into a file on the database specifically for Client X’s WFI Generation system.

6. Off-line samples can be pulled from the WFI Generation system and sent through the MAST system to offline PAT and the LIMS system tracks sample IDs and sample results and saves those results in the database under the same Client X WFI Generation Monitoring file mentioned above.

7. In-line, on-line, at-line, and offline PAT instrument result data are fed to the PAT Software and stored in the client specific file and used to analyze status of the process and produce feed forward and/or feedback control of the unit operations.

8. Specific process parameter limits are saved in the client file and sent to the PCS to control the unit operations while release limits are also sent to SCADA for monitoring.

9. The PAT Software will send predictive feed forward and feedback control commands from the PAT Software to SCADA and finally to the PCS to change process setpoints if needed.

10. Sections a-e above describe the process flow of the WFI Generation or process unit operation specific data flow between hardware and software. Conceptually, above that layer is a digital infrastructure layer. At the center of this layer is the Knowledge Hub which stores historical process and analytical run data. The Knowledge Hub feeds data to process mechanistic models to support digital twins. The digital twin feeds information for process modeling and prediction to the Knowledge Hub, PAT Software, and process monitoring file.

11. A Simulator can also be used to generate optimal process parameters and recipes based on a size and specifications of the connected WFI system.

12. Historical process and analytical raw data from PAT instruments are used to generate libraries from machine learning models that transduce the PAT raw data into quantitative CQA values.

13. Raw data from the PAT instruments is fed to the PAT/PCS control cabinet then through an Application Programming Interface (API) adapter (if needed) which will standardize the communication and data format between the format of the PAT instrument exporting data and the standard format of the PAT Software importing the data.

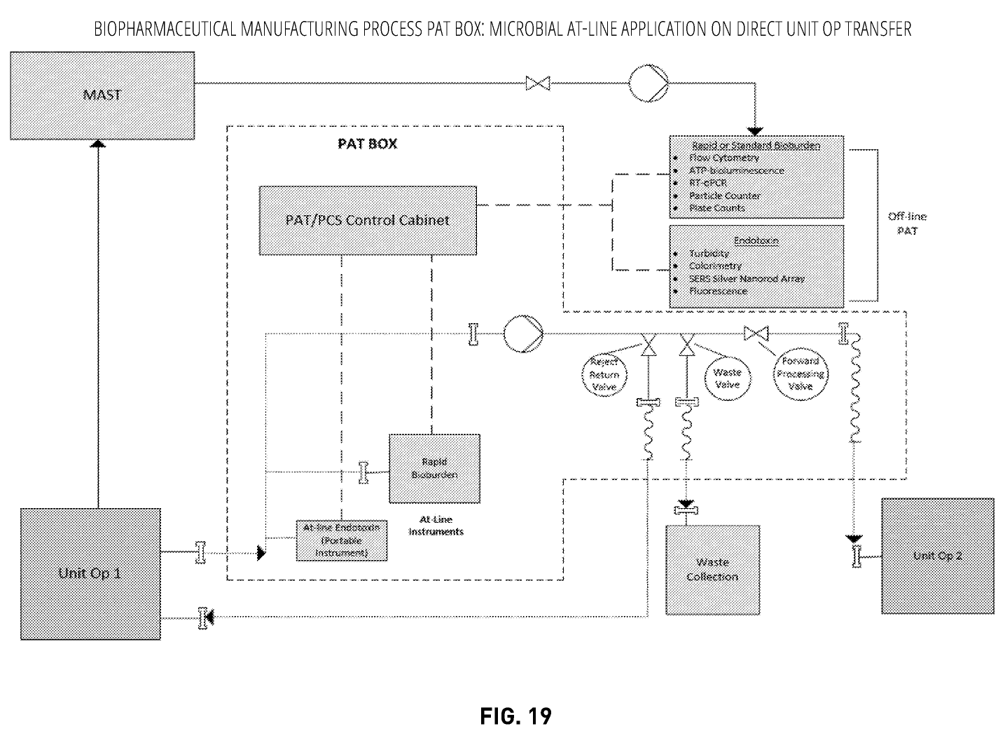

FIG. 19 is a schematic diagram illustrating a PFD of a further developed Microbial PAT Box where the RMM system and the endotoxin instrument are situated in an at-line configuration. The PAT Box boundary is shown by dashed lines and FIG. 19 shows how the PAT Box is situated directly between unit operations with respect to drug manufacturing process systems. The rapid bioburden and endotoxin and offline PAT systems are connected to the PAT/PCS control cabinet so that commands may be sent to the instruments to initiate measurements and result data may be gathered by the PAT/PCS control cabinet and sent to other resources such as the PAT Software for further analysis. This diagram also shows how samples may be taken and sent to an automated sampling system and onward to off-line PAT. Smart valves, including the Reject Return Valve, Waste Valve, and Forward Processing Valve are also depicted showing the ability alter the flow of process material based on the results of the at-line and/or off-line PAT. If bioburden and/or endotoxin results are within pre-defined limits the forward processing valve opens, however if the results do not fall within limits the process material can be sent to waste. A transfer valve and pump can also be used to send the in-process material back to a previous unit operation for further processing if the material does not meet bioburden or endotoxin testing criteria.

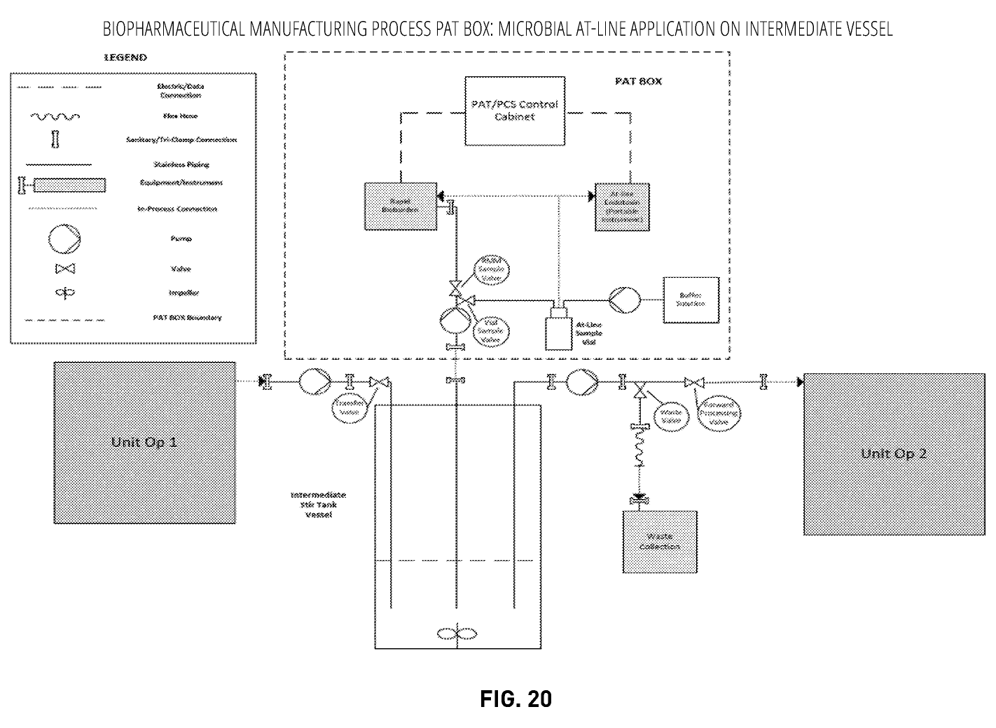

FIG. 20is a schematic diagram illustrating a PFD of Microbial PAT Box where the RMM system and the endotoxin instrument are situated in an at-line configuration. The PAT Box boundary is shown by dashed lines and FIG. 20 shows how the PAT Box is situated where the sample is drawn from an intermediate process vessel that is connected between drug manufacturing process systems Unit Op 1 and Unit Op 2. Examples of these unit operations are chromatography and TFF. This intermediate process vessel serves as a vessel where process material might be held prior to advancing to a further unit operation. The vessel can be sampled into a sample container for transfer to the at-line rapid bioburden and endotoxin systems or directly to the RMM system. The RMM system and the endotoxin system are connected to the PAT/PCS control cabinet which sends commands and receives result data from the RMM and endotoxin instruments. Result data can then be used to command process material to flow from the vessel to either Unit Op 2 or to waste collection.

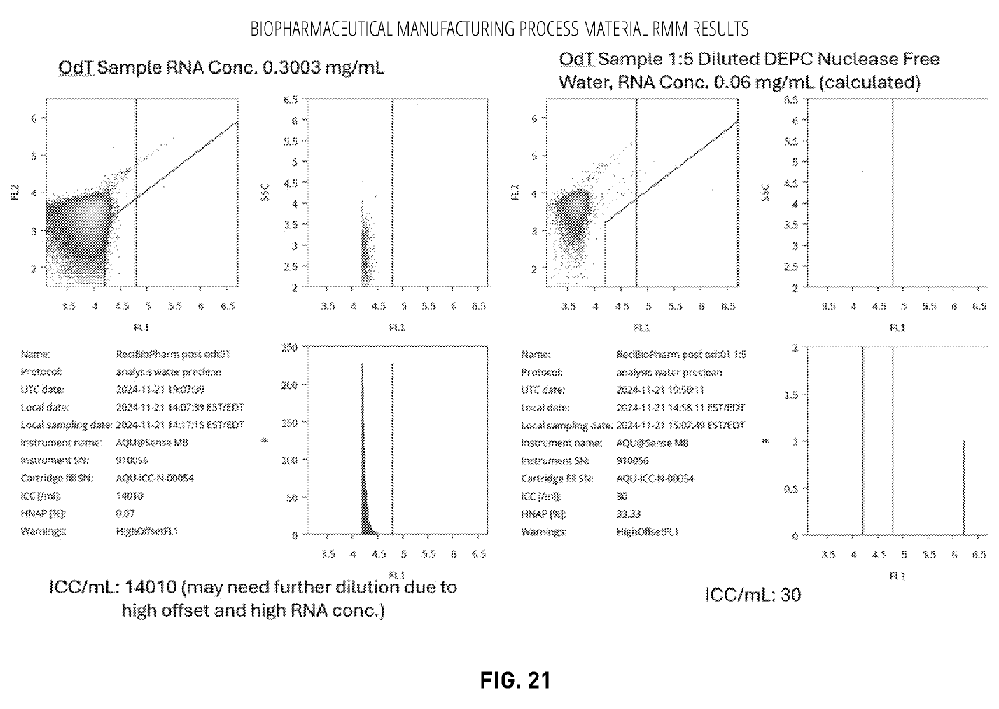

FIG. 21 depicts results of RNA drug substance in-process material (sampled directly after the chromatography unit operation using an OligodT monolith column) measured by an RMM system to determine cell count of viable bioburden cells. The results illustrate the feasibility of using an RMM system with in-process RNA drug substance material. It also shows that further study is needed due to the high offset and signal saturation likely coming from RNA transcripts being tagged with the non-viable cell stain. After further diluting the sample, the fluorescent signal is attenuated to a point where the pre-developed algorithms of the RMM system show the non-viable cell signal and signal coming from the RNA gated on the non-viable side and does not spill over to the viable side of the gate. Further repetition and comparison to off-line results are needed to confirm results. FIG. 21 also shows the possibility that some in-process drug material samples may not be able to be measured in-line due to the concentration of the drug material and may need to be diluted at-line. This application is elucidated in FIG. 19 and FIG. 20.

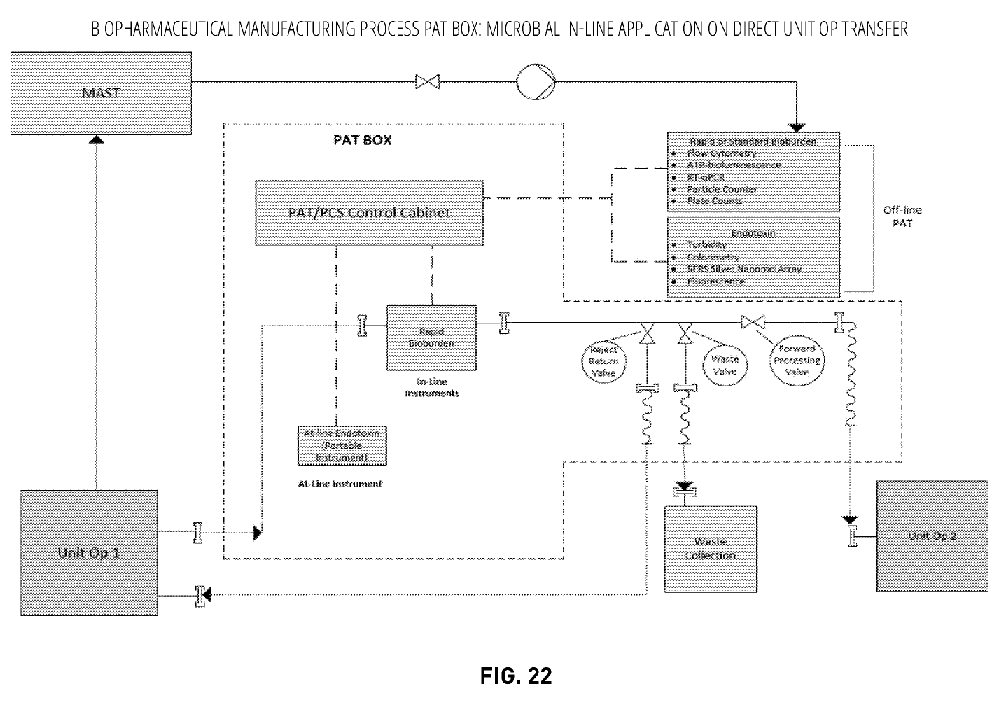

FIG. 22 is a schematic diagram illustrating a PFD of a Microbial PAT Box where the RMM system is situated in-line, and the endotoxin instrument is situated in an at-line configuration. The PAT Box boundary is shown by dashed lines and FIG. 22 shows how the PAT Box is situated directly between unit operations with respect to drug manufacturing process systems. The RMM, endotoxin, and offline PAT systems are connected to the PAT/PCS control cabinet so that commands may be sent to the instruments to initiate measurements and result data may be gathered by the PAT/PCS control cabinet and sent to other resources such as the PAT Software for further analysis. This diagram also shows how samples may be taken and sent to an automated sampling system and onward to off-line PAT. Smart valves, including the Reject Return Valve, Waste Valve, and Forward Processing Valve are also depicted showing the ability alter the flow of process material based on the results of the at-line and/or off-line PAT. Compared to FIG. 19, FIG. 22 shows the RMM system with an in-line configuration in the circumstance that the in-process material is dilute enough to measure without at-line dilution as opposed to the at-line configuration in FIG. 19.

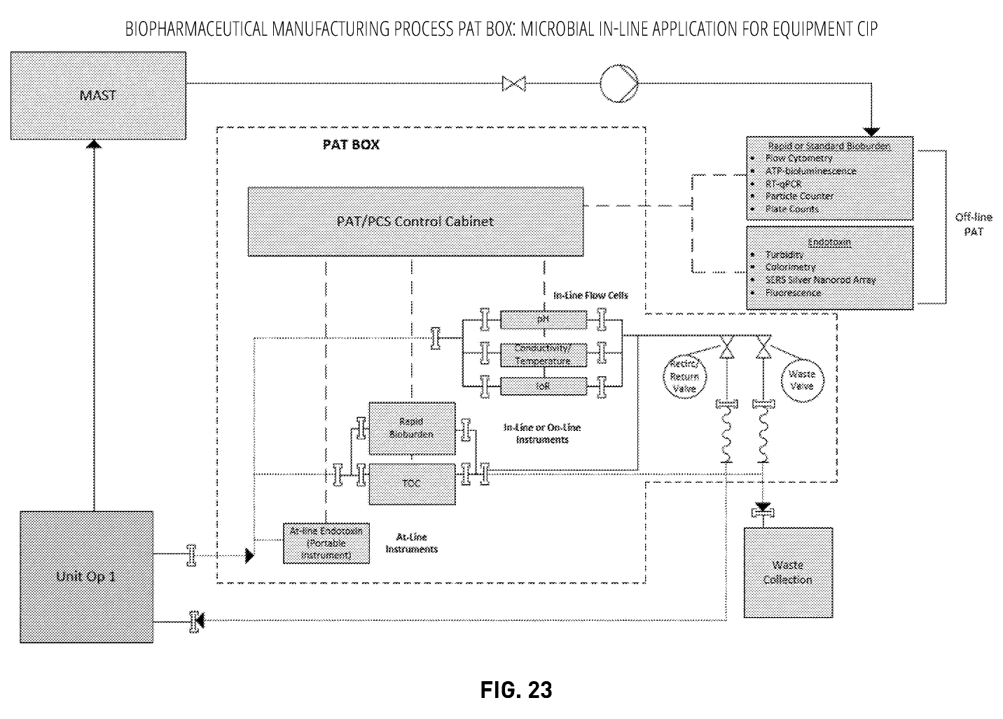

FIG. 23 is a schematic diagram illustrating a PFD of Microbial PAT Box where the RMM system and TOC equipment are both situated either in-line or on-line, while the pH, conductivity, temperature, and IoR instruments are situated in-line, and the endotoxin instrument is situated in an at-line configuration. All PAT instruments are situated on a unit operation system outlet manifold specifically designated for CIP of the unit operation system. The PAT Box boundary is shown by dashed lines and FIG. 23 shows how the PAT Box is directly connected to the CIP outlet of the unit operation and the PAT Box outlet may be connected to either a waste vessel or recirculated back to the unit operation. The in-line, on-line, and at-line PAT instrument systems are connected to the PAT/PCS control cabinet so that commands may be sent to the instruments to initiate measurements and result data may be gathered by the PAT/PCS control cabinet and sent to other resources such as the PAT Software for further analysis. FIG. 23 also shows how samples may be taken and sent to an automated sampling system and onward to off-line PAT. Smart valves, including the Reject Recirc Valve and a Waste Valve are also depicted showing the ability to alter the flow of process material based on the results of the at-line and/or off-line PAT.

FIG. 24 is a schematic diagram illustrating a PFD of an aspect of the mPBS ecosystem specific to controlling and monitoring biopharmaceutical manufacturing unit operation systems with the PAT Box of the mPBS connected directly between unit operations. The diagram also shows the interconnectedness of the PAT Box between the manufacturing systems and the digital infrastructure which drives PAT results analyses and feedback control loops through the PAT Box and into the unit operation systems. FIG. 24 is an analogous figure to FIG. 13 with the difference being the mPBS is situated between two drug manufacturing unit operations as opposed to a WFI generation system. Other differences are a client would input a product construct instead of WFI generation system specifications into the Simulator and the PAT Software would be transducing PAT data for process related CQAs as opposed to WFI generation CQAs

FIG. 25 illustrates an embodiment of a system 6000 such as a PAT management and operations system 702 shown in FIG. 7. The system 6000 is a computer system with multiple processor cores such as a distributed computing system, supercomputer, high-performance computing system, computing cluster, mainframe computer, mini-computer, client-server system, personal computer (PC), workstation, server, portable computer, laptop computer, tablet computer, handheld device such as a personal digital assistant (PDA), or other device for processing, displaying, or transmitting information. Similar embodiments may comprise, e.g., a smart phone or other cellular phone, an external storage device, or the like. Further embodiments implement larger scale server configurations. In other embodiments, the system 6000 may have a single processor with one core or more than one processor. Note that the term “processor” refers to a processor with a single core or a processor package with multiple processor cores.

As shown in FIG. 25, system 6000 comprises a motherboard 6005 for mounting platform components. The motherboard 6005 is a point-to-point interconnect platform that includes a first processor 6010 and a second processor 6030 coupled via a point-to-point interconnect 6056 such as an Ultra Path Interconnect (UPI). In other embodiments, the system 6000 may be of another bus architecture, such as a multi-drop bus. Furthermore, each of processors 6010 and 6030 may be processor packages with multiple processor cores including processor core(s) 6020 and 6040, respectively. While the system 6000 is an example of a two-socket (2S) platform, other embodiments may include more than two sockets or one socket. For example, some embodiments may include a four-socket (4S) platform or an eight-socket (8S) platform. Each socket is a mount for a processor and may have a socket identifier. Note that the term platform refers to the motherboard with certain components mounted such as the processors 6010 and the chipset 6060. Some platforms may include additional components and some platforms may only include sockets to mount the processors and/or the chipset.

The first processor 6010 includes an integrated memory controller (IMC) 6014 and point-to-point (P-P) interconnects 6018 and 6052. Similarly, the second processor 6030 includes an IMC 6034 and P-P interconnects 6038 and 6054. The IMC's 6014 and 6034 couple the processors 6010 and 6030, respectively, to respective memories, a memory 6012 and a memory 6032. The memories 6012 and 6032 may be portions of the main memory (e.g., a dynamic random-access memory (DRAM)) for the platform such as double data rate type 3 (DDR3) or type 4 (DDR4) synchronous DRAM (SDRAM). In the present embodiment, the memories 6012 and 6032 locally attach to the respective processors 6010 and 6030. In other embodiments, the main memory may couple with the processors via a bus and shared memory hub.

The processors 6010 and 6030 comprise caches coupled with each of the processor core(s) 6020 and 6040, respectively. In the present embodiment, the processor core(s) 6020 of the processor 6010 include a PAT management and operations logic 6026 such as the PAT management and operations system 702 shown in FIG. 3. The PAT management and operations logic 6026 may represent circuitry configured to perform configuration, modelling, modeling output within the processor core(s) 6020 or may represent a combination of the circuitry within a processor and a medium to store all or part of the functionality of the PAT management and operations system 6026 in memory such as cache, the memory 6012, buffers, registers, and/or the like. In several embodiments, the functionality of the PAT management and operations logic 6026 resides in whole or in part as code in a memory such as the PAT management and operations logic 6096 in the data storage unit 6088 attached to the processor 6010 via a chipset 6060 such as the PAT Software 512 shown in FIG. 5 or the PAT management and operations system 702 shown in FIG. 7. The functionality of the PAT management and operations logic 6026 may also reside in whole or in part in memory such as the memory 6012 and/or a cache of the processor. Furthermore, the functionality of the PAT management and operations logic 6026 may also reside in whole or in part as circuitry within the processor 6010 and may perform operations, e.g., within registers or buffers such as the registers 6016 within the processor 6010, registers 6036 within the processor 6030, or within an instruction pipeline of the processor 6010 or the processor 6030.

In other embodiments, more than one of the processors 6010 and 6030 may comprise functionality of the PAT management and operations logic 6026 such as the processor 6030 and/or the processor within the deep learning accelerator 6067 coupled with the chipset 6060 via an interface (I/F) 6066. The I/F 6066 may be, for example, a Peripheral Component Interconnect-enhanced (PCI-e).

The first processor 6010 couples to a chipset 6060 via P-P interconnects 6052 and 6062 and the second processor 6030 couples to a chipset 6060 via P-P interconnects 6054 and 6064. Direct Media Interfaces (DMIs) 6057 and 6058 may couple the P-P interconnects 6052 and 6062 and the P-P interconnects 6054 and 6064, respectively. The DMI may be a high-speed interconnect that facilitates, e.g., eight Giga Transfers per second (GT/s) such as DMI 3.0. In other embodiments, the processors 6010 and 6030 may interconnect via a bus.

The chipset 6060 may comprise a controller hub such as a platform controller hub (PCH). The chipset 6060 may include a system clock to perform clocking functions and include interfaces for an I/O bus such as a universal serial bus (USB), peripheral component interconnects (PCIs), serial peripheral interconnects (SPIs), integrated interconnects (I2Cs), and the like, to facilitate connection of peripheral devices on the platform. In other embodiments, the chipset 6060 may comprise more than one controller hub such as a chipset with a memory controller hub, a graphics controller hub, and an input/output (I/O) controller hub.

In the present embodiment, the chipset 6060 couples with a trusted platform module (TPM) 6072 and the unified extensible firmware interface (UEFI), BIOS, Flash component 6074 via an interface (I/F) 6070. The TPM 6072 is a dedicated microcontroller designed to secure hardware by integrating cryptographic keys into devices. The UEFI, BIOS, Flash component 6074 may provide pre-boot code.

Furthermore, chipset 6060 includes an I/F 6066 to couple chipset 6060 with a high-performance graphics engine, graphics card 6065. In other embodiments, the system 6000 may include a flexible display interface (FDI) between the processors 6010 and 6030 and the chipset 6060. The FDI interconnects a graphics processor core in a processor with the chipset 6060.

Various I/O devices 6092 couple to the bus 6081, along with a bus bridge 6080 which couples the bus 6081 to a second bus 6091 and an I/F 6068 that connects the bus 6081 with the chipset 6060. In one embodiment, the second bus 6091 may be a low pin count (LPC) bus. Various devices may couple to the second bus 6091 including, for example, a keyboard 6082, a mouse 6084, communication devices 6086 and a data storage unit 6088 that may store code such as the simulator logic circuitry 6096. Furthermore, an audio I/O 6090 may couple to second bus 6091. Many of the I/O devices 6092, communication devices 6086, and the data storage unit 6088 may reside on the motherboard 6005 while the keyboard 6082 and the mouse 6084 may be add-on peripherals. In other embodiments, some or all the I/O devices 6092, communication devices 6086, and the data storage unit 6088 are add-on peripherals and do not reside on the motherboard 6005.

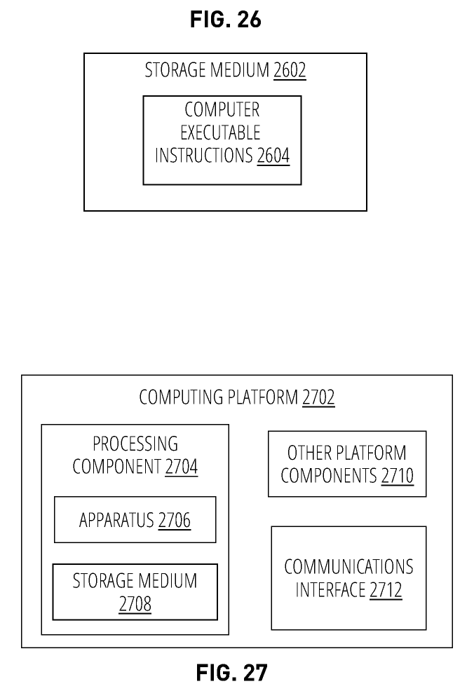

FIG. 26 illustrates an example of a storage medium 2602 to store code for simulator logic circuitry such as the PAT management and operations logic 6096 in the data storage 6088 shown in FIG. 25. Storage medium 2602 may comprise an article of manufacture. In some examples, storage medium 2602 may include any non-transitory computer readable medium or machine readable medium, such as an optical, magnetic or semiconductor storage. Storage medium 2602 may store various types of computer executable instructions, such as instructions to implement logic flows and/or techniques described herein. Examples of a computer readable or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The examples are not limited in this context.

FIG. 27 illustrates an example computing platform 2702 such as the system 6000 shown in FIG. 25. In some examples, as shown in FIG. 27, computing platform 2702 may include a processing component 2704, other platform components or a communications interface 2712. According to some examples, computing platform 2702 may be implemented in a computing device such as a server in a system such as a data center or server farm that supports a manager or controller for managing configurable computing resources as mentioned above. Furthermore, the communications interface 2712 may comprise a wake-up radio (WUR) and may be capable of waking up a main radio of the computing platform 2702.

According to some examples, processing component 2704 may execute processing operations or logic for apparatus 2706 described herein such as the PAT software 512 shown in FIG. 5 or the PAT management and operations system 702 illustrated in FIG. 7. Processing component 2704 may include various hardware elements, software elements, or a combination of both. Examples of hardware elements may include devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements, which may reside in the storage medium 2708, may include software components, programs, applications, computer programs, application programs, device drivers, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given example.

In some examples, other platform components 2710 may include common computing elements, such as one or more processors, multi-core processors, co-processors, memory units, chipsets, controllers, peripherals, interfaces, oscillators, timing devices, video cards, audio cards, multimedia input/output (I/O) components (e.g., digital displays), power supplies, and so forth. Examples of memory units may include without limitation various types of computer readable and machine readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory), solid state drives (SSD) and any other type of storage media suitable for storing information.

In some examples, communications interface 2712 may include logic and/or features to support a communication interface. For these examples, communications interface 2712 may include one or more communication interfaces that operate according to various communication protocols or standards to communicate over direct or network communication links. Direct communications may occur via use of communication protocols or standards described in one or more industry standards (including progenies and variants) such as those associated with the PCI Express specification. Network communications may occur via use of communication protocols or standards such as those described in one or more Ethernet standards promulgated by the Institute of Electrical and Electronics Engineers (IEEE). For example, one such Ethernet standard may include IEEE 802.3-2012, Carrier sense Multiple access with Collision Detection (CSMA/CD) Access Method and Physical Layer Specifications, Published in December 2012 (hereinafter “IEEE 802.3”). Network communication may also occur according to one or more OpenFlow specifications such as the OpenFlow Hardware Abstraction API Specification. Network communications may also occur according to Infiniband Architecture Specification, Volume 1, Release 1.3, published in March 2015 (“the Infiniband Architecture specification”).

Computing platform 2702 may be part of a computing device that may be, for example, a server, a server array or server farm, a web server, a network server, an Internet server, a work station, a mini-computer, a main frame computer, a supercomputer, a network appliance, a web appliance, a distributed computing system, multiprocessor systems, processor-based systems, or combination thereof. Accordingly, functions and/or specific configurations of computing platform 2702 described herein, may be included or omitted in various embodiments of computing platform 2702 , as suitably desired.

The components and features of computing platform 2702 may be implemented using any combination of discrete circuitry, ASICs, logic gates and/or single chip architectures. Further, the features of computing platform 2702 may be implemented using microcontrollers, programmable logic arrays and/or microprocessors or any combination of the foregoing where suitably appropriate. It is noted that hardware, firmware and/or software elements may be collectively or individually referred to herein as “logic”.

It should be appreciated that the computing platform 2702 shown in the block diagram of FIG. 26 may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in embodiments.

One or more aspects of at least one example may be implemented by representative instructions stored on at least one machine-readable medium which represents various logic within the processor, which when read by a machine, computing device or system causes the machine, computing device or system to fabricate logic to perform the techniques described herein. Such representations, known as “IP cores”, may be stored on a tangible, machine readable medium and supplied to various customers or manufacturing facilities to load into the fabrication machines that actually make the logic or processor.

Various examples may be implemented using hardware elements, software elements, or a combination of both. In some examples, hardware elements may include devices, components, processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. In some examples, software elements may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given implementation.

Some examples may include an article of manufacture or at least one computer-readable medium. A computer-readable medium may include a non-transitory storage medium to store logic. In some examples, the non-transitory storage medium may include one or more types of computer-readable storage media capable of storing electronic data, including volatile memory or non-volatile memory, removable or non-removable memory, erasable or non-erasable memory, writeable or re-writeable memory, and so forth. In some examples, the logic may include various software elements, such as software components, programs, applications, computer programs, application programs, system programs, machine programs, operating system software, middleware, firmware, software modules, routines, subroutines, functions, methods, procedures, software interfaces, API, instruction sets, computing code, computer code, code segments, computer code segments, words, values, symbols, or any combination thereof.

According to some examples, a computer-readable medium may include a non-transitory storage medium to store or maintain instructions that when executed by a machine, computing device or system, cause the machine, computing device or system to perform methods and/or operations in accordance with the described examples. The instructions may include any suitable type of code, such as source code, compiled code, interpreted code, executable code, static code, dynamic code, and the like. The instructions may be implemented according to a predefined computer language, manner or syntax, for instructing a machine, computing device or system to perform a certain function. The instructions may be implemented using any suitable high-level, low-level, object-oriented, visual, compiled and/or interpreted programming language.

While the invention herein disclosed has been described by means of specific embodiments and applications thereof, numerous modifications and variations could be made thereto by those skilled in the art without departing from the scope of the invention set forth in the claims.

It will be appreciated that the present invention is set forth in various levels of detail in this application. In certain instances, details that are not necessary for one of ordinary skill in the art to understand the invention, or that render other details difficult to perceive may have been omitted. The terminology used herein is for the purpose of describing particular embodiments only and is not intended to be limiting beyond the scope of the appended claims. Unless defined otherwise, technical terms used herein are to be understood as commonly understood by one of ordinary skill in the art to which the disclosure belongs.

Various features of a process system may be used independently of, or in combination, with each other. It will be appreciated that a system as disclosed herein may be embodied in different forms and should not be construed as limited to the illustrated embodiments of the figures.

It should be understood that, as described herein, an “embodiment” (such as illustrated in the accompanying Figures) may refer to an illustrative representation of an environment or article or component in which a disclosed concept or feature may be provided or embodied, or to the representation of a manner in which just the concept or feature may be provided or embodied. However such illustrated embodiments are to be understood as examples (unless otherwise stated), and other manners of embodying the described concepts or features, such as may be understood by one of ordinary skill in the art upon learning the concepts or features from the present disclosure, are within the scope of the disclosure. In addition, it will be appreciated that while the Figures may show one or more embodiments of concepts or features together in a single embodiment of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one embodiment can be used separately, or with one or more other features to yield a still further embodiment. Thus, it is intended that the present subject matter covers such modifications and variations as come within the scope of the appended claims and their equivalents.

In view of the above, it should be understood that the various embodiments illustrated in the figures have several separate and independent features, which each, at least alone, has unique benefits which are desirable for, yet not critical to, the presently disclosed vessel, system, and associated method. Therefore, the various separate features described herein need not all be present in order to achieve at least some of the desired characteristics and/or benefits described herein.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and / or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader’s understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another.

In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An apparatus of a process analytical technology (PAT) microbial testing skid (mPBS) to capture measurement data for a Critical Quality Attribute (CQA) in real-time comprising:
a vessel for holding a volume of a fluid of a process;
one or more flow cells or immersible probes coupled with one or more instruments to generate the measurement data related to the CQA from the fluid of the process; and
a PAT box cabinet comprising:
the one or more instruments;
an interface for receiving the measurement data; and
a PAT management stack comprising layers of software to manage measurements by the one or more instruments; and
a first controller comprising a processor and memory to execute the layers of software of the PAT management stack and to send the measurement data in a raw or processed form to a second controller to determine the CQA.

2. The apparatus of claim 1, further comprising the second controller, wherein the second controller comprises processing circuitry to execute a model to transform the measurement data in real-time and to output transformed data and trending data related to the CQA and further comprising a human-machine interface (HMI) to present, in real-time, the transformed data and the trending data related to the CQA.

3. The apparatus of claim 1, wherein the one or more instruments comprises up to 10 instruments, wherein the one or more instruments comprise one or more of, or a combination of instruments comprising a rapid microbial method (RMM) analyzer, endotoxin detector, a Total Organic Carbon (TOC) analyzer, a pH meter, conductivity sensor, temperature sensor, and an index of refraction (IoR) system.

4. The apparatus of claim 1, wherein the volume is between 100 milliliters and 100,000 liters, wherein the CQA comprises a volume of a substance, a concentration of the substance, or a mass of the substance, the PAT box cabinet or the vessel to cause the fluid to recirculate through the one or more flow cells and inline probes of the immersible probes to monitor the process and provide real time analytics.

5. The apparatus of claim 4, wherein the vessel is a stirred tank reactor.

6. The apparatus of claim 1, the PAT box cabinet or the vessel to cause the fluid to recirculate through on-line flow cells of the one or more flow cells and sample small aliquots for at-line testing, and, further comprising an automated sampling system to collect samples of the fluid for at-line instruments or off-line instruments, wherein the at-line instruments or the off-line instruments comprises an endotoxin detector.

7. The apparatus of claim 1, wherein the PAT management stack comprises one or more modules, wherein one of the one or modules comprises a calibration and signal processing module to calibrate at least one of the one or more instruments, wherein the calibration and signal processing module is pre-calibrated or automatically calibrated to perform data processing to digitally process the measurement data, wherein the calibration and signal processing module comprises hardware including a flow kit to calibrate or recalibrate at least one of the one or more instruments.

8. The apparatus of claim 2, wherein the second controller comprises an advanced processing controller (APC) to access a library of models to identify and execute one or more models for at least one of the one or more instruments, wherein the APC comprises a chemometrics model and processing circuitry to perform chemometrics to process the measurement data for the at least one of the one or more instruments to transform the measurement data to determine the CQA and trending data related to the at least one of the one or more instruments, wherein the APC comprises or has access to a data storage device for storing the measurement data, the transformed data, and the trending data in a centralized database of PAT measurements and model outputs.

9. The apparatus of claim 8, wherein the APC comprises PAT software to establish one or more parameters for at least one of the one or more instruments, wherein the one or more parameters comprise commanding acquisition parameters or measurement configurations for an in-line instrument, wherein the PAT software comprises or has access to a simulator model to execute in parallel with the process to simulate the process, wherein the simulator model simulates the measurements by the one or more instruments.

10. The apparatus of claim 8, wherein the APC comprises PAT software to perform measurement sequencing, the measurement sequencing including acquiring measurement data, monitoring measurement status, raising errors if measurements fail, storing raw measurement data, transferring raw measurement data to a model execution web app, receiving transformed data and model outputs from the model execution web app, and storing transformed data and model outputs, wherein the APC comprises PAT software to enable execution of Python models in a model execution web app, wherein the model execution web app receives the data from the measurement sequencing and runs Python codes to process measurement data, wherein the model execution web app executes any Python models that can handle real-time data, wherein one or more of the Python models comprise pre-treatment methods including interpolating functions, derivation, and normalization; multivariate models including principal component analysis (PCA) and partial least square (PLS) regression; and hybrid models by combining mechanistic equations and the multivariate models, wherein the APC comprises PAT software enable concurrent operation of multiple instruments and models.

11. A method a process analytical technology (PAT) microbial testing skid (mPBS) to capture measurement data for a Critical Quality Attribute (CQA) in real-time comprising:
manage measurements by one or more instruments;
generating measurement data from a process in a vessel holding a volume of a fluid via one or more flow cells or immersible probes coupled with the one or more instruments, the measurement data related to the CQA from the fluid of the process;
receiving the measurement data; and
processing, via a PAT management stack of a first controller, the measurement data; and
sending the measurement data in a raw form, a processed form, or a combination thereof, to a second controller to determine the CQA.

12. The method of claim 11, further comprising executing a model, via the second controller, to transform the measurement data in real-time and to output transformed data and trending data related to the CQA and further comprising displaying, via a human-machine interface (HMI), in real-time, the transformed data and the trending data related to the CQA.

13. The method of claim 11, wherein the one or more instruments comprises up to 10 instruments, wherein the one or more instruments comprise one or more of, or a combination of instruments comprising a rapid microbial method (RMM) analyzer, endotoxin detector, a Total Organic Carbon (TOC) analyzer, a pH meter, conductivity sensor, temperature sensor, and an index of refraction (IoR) system.

14. The method of claim 11, wherein the volume is between 100 milliliters and 100,000 liters, wherein the CQA comprises a volume of a substance, a concentration of the substance, or a mass of the substance, and recirculating the fluid through the one or more flow cells and inline probes of the immersible probes to monitor the process and provide real time analytics.

15. The method of claim 11, wherein the vessel is a stirred tank reactor.

16. The method of claim 11, further comprising sampling small aliquots of the fluid for at-line testing and collecting, via an automated sampling system, samples of the fluid for at-line instruments or off-line instruments, wherein the at-line instruments or the off-line instruments comprises an endotoxin detector.

17. The method of claim 11, further comprising calibrating, via a calibration and signal processing module of the PAT management stack, at least one of the one or more instruments and digitally processing the measurement data via the calibration and signal processing module, wherein the calibration and signal processing module is pre-calibrated or automatically calibrated to digitally process the measurement data, wherein the calibration and signal processing module comprises hardware including a flow kit to calibrate or recalibrate at least one of the one or more instruments.

18. The method of claim 12, further comprising accessing a library of models to identify and execute one or more models for at least one of the one or more instruments via the second controller, wherein the second controller comprises an advanced processing controller (APC); performing chemometrics, via the APC, to process the measurement data for the at least one of the one or more instruments to transform the measurement data to determine the CQA and trending data related to the at least one of the one or more instruments; and accessing, via the APC, a data storage device for storing the measurement data, the transformed data, and the trending data in a centralized database of PAT measurements and model outputs.

19. The method of claim 18, further comprising:
establishing one or more parameters for at least one of the one or more instruments via PAT software of the APC, wherein establishing the one or more parameters comprises commanding acquisition parameters or measurement configurations for an in-line instrument;
executing, via the PAT software, a simulator model in parallel with the process to simulate the process executing the simulator model simulates the measurements by the one or more instruments;
performing, via the PAT software of the APC, measurement sequencing, the measurement sequencing including acquiring measurement data, monitoring measurement status, raising errors if measurements fail, storing raw measurement data, transferring raw measurement data to a model execution web app, receiving transformed data and model outputs from the model execution web app, and storing transformed data and model outputs;
enabling, via the PAT software of the APC, execution of Python models in a model execution web app; receiving, via the model execution web app, the data from the measurement sequencing and running Python codes to process measurement data;
executing, via the model execution web app, any Python models that can handle real-time data, wherein one or more of the Python models comprise pre-treatment methods including interpolating functions, derivation, and normalization; multivariate models including principal component analysis (PCA) and partial least square (PLS) regression; and hybrid models by combining mechanistic equations and the multivariate models; and
enabling, via the PAT software of the APC, concurrent operation of multiple instruments and models.

20. A system of a process analytical technology (PAT) microbial testing skid (mPBS) to capture measurement data for a Critical Quality Attribute (CQA) in real-time comprising:
a vessel for holding a volume of a fluid of a process;
one or more flow cells or immersible probes coupled with one or more instruments to generate the measurement data related to the CQA from the fluid of the process; and
a PAT box cabinet comprising:
the one or more instruments;
an interface for receiving the measurement data; and
a PAT management stack comprising layers of software to manage measurements by the one or more instruments;
a first controller comprising a processor and memory to execute the layers of software of the PAT management stack and to send the measurement data in a raw or processed form to a second controller to determine the CQA; and
the second controller, wherein the second controller comprises processing circuitry to execute a model to transform the measurement data in real-time and to output transformed data and trending data related to the CQA.

21. The system of claim 20, further comprising:
a human-machine interface (HMI) to present, in real-time, the transformed data and the trending data related to the CQA, wherein the one or more instruments comprises up to 10 instruments, wherein the one or more instruments comprise one or more of, or a combination of instruments comprising a rapid microbial method (RMM) analyzer, endotoxin detector, a Total Organic Carbon (TOC) analyzer, a pH meter, conductivity sensor, temperature sensor, and an index of refraction (IoR) system, wherein the CQA comprises a volume of a substance, a concentration of the substance, or a mass of the substance.

22. The system of claim 20, further comprising an automated sampling system to collect samples of the fluid for at-line instruments or off-line instruments, wherein the at-line instruments or the off-line instruments comprises an endotoxin detector.

23. The system of claim 20, wherein the second controller comprises an advanced processing controller (APC) to access a library of models to identify and execute one or more models for at least one of the one or more instruments, wherein the APC comprises a chemometrics model and processing circuitry to perform chemometrics to process the measurement data for the at least one of the one or more instruments to transform the measurement data to determine the CQA and trending data related to the at least one of the one or more instruments.

24. The system of claim 23, wherein the APC comprises PAT software to establish one or more parameters for at least one of the one or more instruments, wherein the one or more parameters comprise commanding acquisition parameters or measurement configurations for an in-line instrument, wherein the PAT software comprises or has access to a simulator model to execute in parallel with the process to simulate the process, wherein the simulator model simulates the measurements by the one or more instruments, wherein the APC comprises PAT software to perform measurement sequencing, the measurement sequencing including acquiring measurement data, monitoring measurement status, raising errors if measurements fail, storing raw measurement data, transferring raw measurement data to a model execution web app, receiving transformed data and model outputs from the model execution web app, and storing transformed data and model outputs.
